(19) United States
(12) Patent Application Publication
(10) Pub. No.: US 2008/0025940 A1
(43) Pub. Date: Jan. 31, 2008

(54) Administration of 3-sulfanylpropanamide compounds for stimulating hair and eyelash growth and/or for stopping the loss and/or limiting the depigmentation thereof

(75) Inventors: Philippe Breton, Noisy Le Roi (FR); Christophe Boulle, Lagny S/Marne (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 11/882,331

(22) Filed: Jul. 31, 2007

(30) Foreign Application Priority Data
Jan. 31, 2005 (FR) 0550279
Jan. 23, 2006 (EP) PCT/EP2006/001545

(51) Int. Cl. A61K 31/165; A61K 31/341; A61K 31/4192; A61K 8/46; A61K 8/49; A61Q 5/00; A61Q 7/00; C07C 233/05; C07D 249/04; C07D 307/54

(52) U.S. Cl. 424/70.7; 424/70.1; 514/359; 514/471; 514/625; 548/261; 549/493; 564/192

(57) ABSTRACT

3-Sulfanylpropanamide compounds of formula (I) or salts and/or solvates thereof: 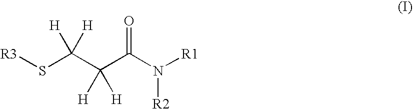 are useful for caring for and/or making up keratin fibers, especially human keratin fibers, to induce and/or stimulate the growth, to stop the loss and/or to increase the density thereof, and/or to improve the appearance and/or to reduce the canities thereof, and/or to stimulate the pigmentation and/or to limit the depigmentation and/or bleaching thereof.

### CROSS-REFERENCE TO PRIORITY/PCT/PROVISIONAL APPLICATIONS

This application claims priority under 35 U.S.C. § 119 of FR 05/50279, filed Jan. 31, 2005, and of Provisional Application No. 60/670,020, filed Apr. 11, 2005, and is a continuation of PCT/EP 2006/001545 filed Jan. 23, 2006 and designating the United States, published in the English language as WO 2006/079568 A3 on Aug. 3, 2006, each hereby expressly incorporated by reference in its entirety and each assigned to the assignee hereof.

### BACKGROUND OF THE INVENTION

1. Technical Field of the Invention

The present invention relates to the administration of compositions comprising 3-sulfanylpropanamide compounds for promoting and/or inducing and/or stimulating the pigmentation of keratin fibers and also for limiting their depigmentation and/or bleaching.

This invention also relates to novel 3-sulfanylpropanamide compounds and to compositions comprising same that are useful for increasing the density and/or improving the appearance of the said keratin fibers and/or for reducing canities thereof.

The present invention also relates to a cosmetic treatment regime or regimen for stimulating the growth of keratin fibers and/or for stopping their loss and/or for stimulating their pigmentation and/or for limiting their depigmentation and/or bleaching.

The human keratin fibers to which this invention applies are especially head hair, the eyebrows, the eyelashes, beard hairs, moustache hairs and pubic hairs. More especially, this invention relates to human head hair and/or eyelashes. The present invention also relates to human nails.

2. Description of Background and/or Related and/or Prior Art

Hair growth and hair renewal are mainly determined by the activity of the hair follicles and of their matrix environment. Their activity is cyclical and comprises essentially three phases, namely, the anagenic phase, the catagenic phase and the telogenic phase.

The anagenic phase (active phase or growth phase), which lasts several years and during which the hair gets longer, is followed by a very short and transient catagenic phase that lasts a few weeks. During this phase, the hair undergoes a change, the follicle becomes atrophied and its dermal implantation appears higher and higher.

The terminal phase or telogenic phase, which lasts a few months, corresponds to a resting phase of the follicle and the hair ultimately falls out. At the end of this rest period, a new follicle is regenerated in situ and another cycle begins.

The head of hair is thus under permanent renewal, and, out of the approximately 150,000 hairs that make up a head of hair, about 10% are at rest and will be replaced within a few months.

The natural loss or falling-out of the hair may be estimated, on average, as being a few hundred hairs per day for a normal physiological state. This process of permanent physical renewal undergoes a natural change during aging, the hairs become finer and their cycles shorter.

In addition, various causes may result in a substantial, temporary or permanent loss of hair. This may be loss and impairment of hair at the terminal stage of pregnancy (postpartum), during states of dietary malnutrition or imbalance, during physiological stress, or during states of asthenia or of hormonal dysfunction, as may be the case during or at the terminal stage of the menopause. It may also be a case of loss or impairment of the hair related to seasonal phenomena.

It may also be a matter of alopecia, which is essentially due to a disturbance in hair renewal, resulting, in a first stage, in acceleration of the frequency of the cycles to the detriment of the quality of the hair, and then of their quantity. The successive growth cycles result in hairs that are finer and finer and shorter and shorter, gradually transforming into an unpigmented down, thus resulting in a progressive impoverishment of the head of hair. Certain areas are preferentially affected, especially the temporal or frontal lobes in men, and a diffuse alopecia of the crown of the head in women.

The term alopecia also covers a whole family of afflictions of hair follicles whose final consequence is the permanent, partial or general loss of the hair. This is more particularly a matter of androgenic alopecia. In a large number of cases, early loss of hair occurs in genetically predisposed individuals; this is then a matter of androchronogenetic alopecia. This form of alopecia especially affects men.

In certain dermatoses of the scalp with an inflammatory component, for instance psoriasis or seborrhoeic dermatitis, hair loss may be greatly accentuated or may result in highly disrupted follicular cycles.

The cosmetics and pharmaceutical industries have for many years been investigating compositions for eliminating or reducing alopecia, and especially for inducing or stimulating hair growth or reducing its loss.

In this perspective, a large number of compositions comprising very diverse active agents have already been proposed, for instance 2,4-diamino-6-piperidinopyrimidine 3-oxide, or “minoxidil”, described in U.S. Pat. Nos. 4,139,619 and 4,596,812, or the numerous derivatives thereof such as those described in EP-0,353,123, EP-0,356,271, EP-0,408,442, EP-0,522,964, EP-0,420,707, EP-0,459,890 and EP-0,519,819.

Clinical studies have shown that PGF2-α analogs have the property of inducing the growth of body hairs and eyelashes in man and animals (Murray A. and Johnstone M. D., 1997, Am. J. Opht., 124(4), 544-547). In man, tests performed on the scalp have shown that a prostaglandin E2 analogue (viprostol) has the property of increasing the hair density (Roenigk H. H., 1988, Clinic Dermatol., 6(4), 119-121).

Moreover, WO 98/33497 describes pharmaceutical compositions containing prostaglandins or prostaglandin derivatives, for combating hair loss in man. Prostaglandins of the type A2, F2α and E2 are mentioned as being preferred.

However, prostaglandins are molecules with a very short biological half-life, which act in an autocrine or paracrine manner, this reflecting the local and labile nature of the metabolism of prostaglandins (Narumiya S. et al., 1999, Physiol. Rev., 79(4), 1193-1226).

It is thus seen to be important, in order to maintain and/or increase the hair density in man, to preserve the endogenous reserves of PGF2-α and similarly of PGE2 in various compartments of the hair follicle or its immediate cutaneous environment.

One solution that provides good results is the administration of lipoxygenase-inhibiting compounds and/or cyclooxygenase-inducing compounds to promote hair growth; one theory is that administration of such compounds directs the metabolism of fatty acids towards the endogenous synthesis of prostaglandins in preference to other routes.

However, to further improve these results, it would be desirable to prolong the activity of the prostaglandins involved in growing the hair and keeping it alive.

It is moreover well known that the programs of differentiation of the keratinocytes of the epidermis and of the hair follicle are clearly different. Thus, it is known that the keratins of the hair shaft are each a family (Langbein et al., 2001, J. Biol. Chem., 276: 35123-35132) that is different from the one expressed in the epidermis, that differentiation markers such as the keratins K₁ and K₁₀ are not expressed in the hair follicle and in particular in the outer sheath (Lenoir et al., 1988, Dev. Biol. 130: 610-620), that trichohyalin (O'Guin et al., 1992, J. Invest. Dermatol., 98: 24-32) and keratin K6irs (Porter et al., 2001, Br. J. Dermatol., 145: 558-568) are expressed in the hair follicle, in particular in the inner sheath, but not in the epidermis, and that type-1 cyclooxygenase, although expressed in the epidermis, is not expressed in the keratinocytes of the hair follicle but in the dermal papilla (Michelet. et al., 1997, J. Invest. Dermatol., 108: 205-209).

The assignee hereof has now demonstrated that an enzyme specifically involved in the degradation of these prostaglandins is present in the dermal papilla of the hair, which is a compartment that is a decisive factor in the life of the hair. Specifically, the assignee hereof has now proven the presence of type-1 15-hydroxyprostaglandin dehydrogenase (abbreviated to 15-PGDH) at this level. The assignee hereof has also shown that the inhibition of type-1 15-PGDH has a beneficial effect on hair growth (see WO 03/090699).

Type-1 15-PGDH is a key enzyme in the deactivation of prostaglandins, in particular of PGF2-α and PGE2, which are important mediators of hair growth and survival. It corresponds to the classification EC 1.1.1.141 and is NAD+-dependent. This enzyme catalyzes an oxidation reaction on carbon 15 of the hydroxyl into ketone. It has been isolated from pig kidney; its inhibition with a thyroid hormone, triiodothyronine, at doses very much higher than the physiological doses, has especially been observed. Type-2 15-PGDH is itself NADP-dependent.

### SUMMARY OF THE INVENTION

However, it has never been proposed to administer a type-1 15-PGDH inhibitor to maintain and/or increase the density of human keratin fibers and especially the hair density and/or to reduce the heterogeneity of the diameters of the keratin fibers and especially of the hair in man. The expression “increase the density of human keratin fibers, and especially the hair density” means increasing the number of keratin fibers, and especially of hairs per cm² of skin or of scalp.

Consequently, the present invention features administration of compositions comprising at least one particular 15-hydroxyprostaglandin dehydrogenase inhibitor for promoting the growth of human keratin fibers such as head hair and/or other bodily hairs.

Moreover, this invention also features novel products for caring for and/or treating human keratin fibers, but also for human skin, which promotes their pigmentation and/or limits their depigmentation and which makes it possible in particular to prevent and/or reduce the canities of human keratin fibers such as head hair, the eyelashes and/or certain other bodily hairs.

The color of human hair and skin depends on various factors and especially on the seasons of the year, race, sex and age. It is mainly determined by the concentration of melanin produced by the melanocytes. These melanocytes are specialized cells that synthesize melanin via particular organelles, the melanosomes.

Melanin synthesis (or melanogenesis) is complex and schematically involves the following main steps:
Tyrosine->Dopa->Dopaquinone->Dopachrome->Melanin

Tyrosinase (monophenol dihydroxyl phenylalanine:oxygen oxidoreductase EC 1.14.18.1) participates in this sequence of reactions by especially catalyzing the reaction for conversion of tyrosine into dopa (dihydroxyphenylalanine) and the reaction for conversion of dopa into dopaquinone.

The upper part of the hair follicle appears as a tubular invagination of the epidermis, which is buried just down to the deep layers of the dermis. The lower part, or hair bulb, itself comprises an invagination in which is found the dermal papilla. Around the dermal papilla, in the lower part of the bulb, is an area populated with cells with a high degree of proliferation (matrix cells). These cells are the precursors of the keratinized cells that will constitute the hair. The cells that result from the proliferation of these precursors migrate vertically in the bulb and become gradually keratinized in the upper part of the bulb; this assembly of keratinized cells will form the hair stem. Pigmentation of the hair and of other bodily hairs requires the presence of melanocytes in the bulb of the hair follicle. These melanocytes are in an active state, i.e., they synthesize melanins (or melanin pigments). These pigments are transferred to the keratinocytes intended to form the hair stem, which will give rise to the growth of a pigmented head hair or other bodily hair. This structure is known as a “follicular pigmentation unit”.

It is known that, in the majority of populations, a brown skin coloration and maintenance of a constant coloration of head hair are important aspirations.

It is accepted that the appearance of grey or white bodily hairs and/or head hairs, or canities, is associated with a decrease in melanin in the hair stem. This phenomenon occurs naturally during the life of an individual. However, people are seeking to have a more youthful appearance and, with an aesthetic goal, they are often tempted to combat this phenomenon, especially when it occurs at a relatively early age.

Many solutions have thus been proposed in the field of artificial coloration by providing exogenous dyes intended to give the hair a coloration as close as possible to that which it has naturally. Another approach entails stimulating the natural pigmentation pathway.

Among the proposed solutions, exemplary are compositions containing a phosphodiesterase inhibitor (WO 95/17161), DNA fragments (WO 95/01773), diacylglycerol (WO 94/04122), prostaglandins (WO 95/11003) or pyrimidine 3-oxide derivatives (EP-829,260).

It has now unexpectedly been found that it is possible to stimulate the synthesis of melanin by the melanocytes by specifically inhibiting the degradation of the prostaglandins synthesized by these melanocytes or those present in its environment.

Accordingly, the present invention features compositions comprising at least one particular 15-hydroxyprostaglandin dehydrogenase inhibitor for promoting the pigmentation of the skin or the integuments, in particular human keratin fibers such as head hair and/or other bodily hairs.

The involvement of certain prostaglandins in the pigmentation of bodily hairs or the skin in man or animals is described in Wand M., 1997, Arch. Ophtalmol., 115; Abdel Malek et al., 1987, Cancer Res., 47. However, since prostaglandins are molecules with a very short biological half-life and as a result of the local and labile nature of their metabolism (Narumiya S. et al.), it appears important to be able to prolong the activity of the prostaglandins involved in the pigmentation of human skin, bodily hairs and/or head hair.

Moreover, the assignee hereof has shown that 15-hydroxyprostaglandin dehydrogenase is also expressed in the melanocyte of head hairs, which had never been demonstrated hitherto (see WO 04/073 594). In addition, the assignee hereof has demonstrated the presence of 15-PGDH in the dermal papilla and the melanocyte of head hairs, and has proposed administering a 15-PGDH inhibitor to promote the pigmentation of human skin, bodily hairs and/or head hair. It is now possible to locally regulate the level of prostaglandins and especially that present in the melanocyte, in particular of head hair, by acting on the degradation catalyzed both by the 15-PGDH of the melanocyte and of the fibroblast of the dermal papilla.

The assignee hereof has also shown that the melanocytes of head hairs express prostaglandin H synthase 1 (PGHS-1 or COX-1, E.C.: 1.14.99.1). This demonstrates for the first time that the melanocytes of head hair have an autonomous metabolism of prostaglandins.

In WO 04/073 594, it was furthermore shown that it is possible to specifically inhibit the 15-PGDH present in the dermal papilla and/or in the melanocyte of head hair. Such an inhibition thus makes it possible to stop the deactivation of the prostaglandins in the environment of the melanocyte of head hair. The prostaglandins can thus continue via the autocrine or paracrine route to stimulate the melanocytes. Specifically, the application of such inhibitors stimulates the production of melanin by the melanocytes.

The present invention thus also features compositions for caring for and/or treating the skin and/or keratin fibers, especially human skin and/or keratin fibers such as the hair, containing at least one particular 15-hydroxyprostaglandin dehydrogenase inhibitor, especially of type 1 and a physiologically acceptable medium.

According to the invention, the term “15-PGDH inhibitor” means any substance, simple or complex compound, of natural or synthetic origin, capable of inhibiting or of reducing the activity of the enzyme 15-PGDH, and/or capable of inhibiting, reducing or slowing down the reaction catalyzed by this enzyme. The 15-PGDH inhibitors according to the invention are preferably type-1 15-PGDH inhibitors.

Advantageously, the inhibitor is a specific NAD-dependent type-1 15-PGDH inhibitor.

Also, surprisingly, it has now been found that certain salified or non-salified, solvated or non-solvated 3-sulfanylpropanamide derivatives are inhibitors of 15-hydroxyprostaglandin dehydrogenase, in particular of type 1. It has moreover been found that these compounds have favorable activity on increasing the density of human keratin fibers and/or on limiting the canities thereof.

### DETAILED DESCRIPTION OF BEST MODE AND SPECIFIC/PREFERRED EMBODIMENTS OF THE INVENTION

The present invention features the administration, especially the cosmetic administration, of at least one 3-sulfanylpropanamide compound of formula (I) or a salt and/or solvate thereof: 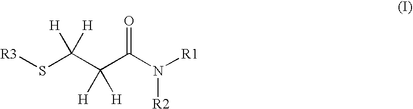
in which:
a) R₁ and R₂ independently represent:
   1) a hydrogen atom,
   2) a C₁-C₂₀ alkyl radical optionally substituted with at least one substituent A₁,
   3) a saturated hydrocarbon-based ring C¹ optionally substituted with at least one substituent A₁;
   4) an unsaturated hydrocarbon-based ring C² optionally substituted with at least one substituent A₂;
b) R₃ is:
   1) a hydrogen atom,
   2) a C₁-C₂₀ alkyl radical optionally substituted with at least one substituent A₃,
   3) a saturated hydrocarbon-based ring C³ optionally substituted with at least one substituent A₄;
c) A₁ is:
   1) a fluorine atom,
   2) a group CF₃, CN, OR, SR, NRR′, SO₂NRR′, NRSO₂R′ or SO₂R,
   3) a group having the following formula (II): 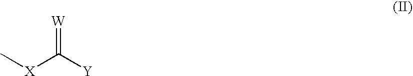
      wherein:
   W=O, NH or S,
   X=O, NH, C₁-C₆ N-alkyl,
   Y=C₁-C₄ alkyl, C₁-C₆ alkoxy, NH₂, C₁-C₆ NH-alkyl, C₁-C₆ N-dialkyl,
   4) a saturated hydrocarbon-based ring C⁴ optionally substituted with at least one substituent A⁴,
   5) a heterocycle Hy₁ selected from the group consisting of pyrrole, furan, dihydrofuran, tetrahydrofuran, thiophene, thiazole, oxazole, oxazolidine, isoxazole, isoxazolidine, oxadiazole, pyridine, pyran, dihydropyran, tetrahydropyran, pyrimidine, piperazine, pyridazine, pyrazine, morpholine, quinoline, isoquinoline, 2-thiohydantoin, 2-oxo-2,3-dihydro-1,3-thiazole and pyridone, such heterocycle optionally being substituted with at least one substituent A₄,
   6) an unsaturated hydrocarbon-based ring C³ optionally substituted with at least one substituent A₄ and optionally fused to a hydrocarbon-based or heterocyclic ring C⁶;
d) A₂ is:
   1) a fluorine atom,
   2) a group of formula CF₃, CN, OR, SR, NRR′, COR, COOR, CONRR′,NRCOR′,NRCONR′R″,SO₂NRR′, NRSO₂R′ or SO₂R,
   3) a C₁-C₂₀ alkyl radical optionally substituted with at least one substituent A₄;
e) A₃ is:
   1) a fluorine atom,
   2) a group CF₃, CN, OR, SR, NRR′,
   3) a group of formula (II) 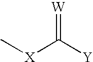
      wherein W, X and Y have the same definitions as above,
   4) a saturated hydrocarbon-based ring C⁴ optionally substituted with at least one substituent A₄,
   5) an unsaturated hydrocarbon-based ring C⁵ optionally substituted with at least one substituent A₄ or a heterocycle Hy₂ optionally substituted with at least one substituent A₄ and containing at least one hetero atom selected from among N, O and S and combinations thereof;
f) A₄is:
   1) a fluorine atom,
   2) a group CF₃, CN, OR, SR, NRR′,
   3) a C₁-C₂₀ alkyl radical, or
   4) a ring C⁶ optionally fused to another ring C⁷, these rings optionally containing at least one hetero atom to form a heterocycle Hy₂;
g) R, R′ and R″, which may be identical or different, represent:
   1) a hydrogen atom,
   2) a saturated C₁-C₆ alkyl radical optionally substituted with at least one substituent A₄, or
   3) an unsaturated hydrocarbon-based ring C⁵ optionally substituted with at least one substituent A₄,
   4) a heterocycle Hy₂ comprising at least one hetero atom selected from among N, S and O or a combination thereof and optionally being substituted with at least one substituent A₄.

The present invention also features the administration, especially the cosmetic administration, of at least one 3-sulfanylpropanamide compound of formula (I) or a salt and/or solvate thereof, as defined above, as an agent for promoting and/or inducing and/or stimulating the pigmentation of the skin and/or keratin fibers, especially human skin and/or keratin fibers, and/or as an agent for preventing and/or limiting the depigmentation and/or bleaching of the skin and/or keratin fibers, especially human skin and/or keratin fibers, and, especially as an agent for preventing and/or limiting the canities of human keratin fibers.

This invention also features the formulation of at least one 3-sulfanylpropanamide compound of formula (I) or a salt and/or solvate thereof into a cosmetic care and/or makeup composition for keratin fibers, especially human keratin fibers, to induce and/or stimulate their growth, to stop their loss and/or to increase their density.

This invention also features the formulation of at least one 3-sulfanylpropanamide compound of formula (I) or a salt and/or solvate thereof into a cosmetic care and/or makeup composition for the skin and/or keratin fibers, especially human skin and/or keratin fibers, to induce and/or stimulate the pigmentation of the said skin and/or the said fibers and/or to limit their depigmentation and/or bleaching.

The present invention also features the formulation of at least one 3-sulfanylpropanamide compound of formula (I) or a salt and/or solvate thereof into a care or treatment composition for keratin fibers, especially human keratin fibers, which compositions are useful to induce and/or stimulate the growth of the said fibers and/or to stop their loss and/or to increase their density.

The present invention also features the formulation of at least one 3-sulfanylpropanamide compound of formula (I) or a salt and/or solvate thereof into a care or treatment composition for the skin and/or keratin fibers, especially human skin and/or keratin fibers, which compositions are useful to induce and/or stimulate the pigmentation of the skin and/or the said keratin fibers and/or to limit the depigmentation and/or bleaching of the skin and/or keratin fibers, and which is especially useful to prevent and/or limit the bleaching or canities of human keratin fibers.

This invention also features the formulation of at least one 3-sulfanylpropanamide compound of formula (I) or a salt and/or solvate thereof into a cosmetic care and/or makeup composition for keratin fibers, especially human keratin fibers, as an agent for reducing their loss and/or for increasing their density and/or for limiting their bleaching. Such compositions also make it possible to maintain the keratin fibers in good condition and/or to improve their appearance.

The present invention is also applicable to the keratin fibers of mammals of the animal species (for example dog, horse or cat).

The human keratin fibers to which this invention applies are especially head hair, the eyebrows, the eyelashes, beard hairs, moustache hairs and pubic hairs, and also the nails. More especially, the present invention applies to human head hair and/or eyelashes.

Thus, this invention also features the formulation of at least one 3-sulfanylpropanamide compound of formula (I) or a salt and/or solvate thereof, into a cosmetic composition for human haircare to reduce the loss of the hair and/or to increase its density and/or to treat androchronogenetic alopecia and/or to reduce its depigmentation and/or bleaching.

This invention also features the formulation of at least one 3-sulfanylpropanamide compound of formula (I) or a salt and/or solvate thereof into a human hair composition, which composition is useful to induce and/or stimulate the growth of the hair and/or to stop its loss and/or to increase its density and/or to treat androgenic alopecia and/or to induce and/or stimulate its pigmentation and/or to limit its depigmentation and/or bleaching.

This invention also features the formulation of at least one 3-sulfanylpropanamide compound of formula (I) or a salt and/or solvate thereof, into a cosmetic composition for human haircare to treat alopecia of natural origin and in particular androgenic alopecia, and also the formulation of at least one 3-sulfanylpropanamide compound of formula (I) or a salt and/or solvate thereof into a human haircare composition for treating alopecia of natural origin and in particular androgenic alopecia. Thus, this composition makes it possible to maintain the hair in good condition and/or to combat the natural loss of the hair and more especially that of men.

The present invention also features the formulation of at least one 3-sulfanylpropanamide compound of formula (I) or a salt and/or solvate thereof, into a cosmetic composition for caring for and/or making up human eyelashes, to induce and/or stimulate the growth of the eyelashes and/or to increase their density and/or to limit their bleaching, and also to formulating at least one 3-sulfanylpropanamide compound of formula (I) or a salt and/or solvate thereof, into a composition for caring for and/or treating human eyelashes, which composition is useful to induce and/or stimulate the growth of the eyelashes and/or to increase their density and/or to induce and/or stimulate their pigmentation and/or to limit their depigmentation and/or bleaching. This composition thus makes it possible to maintain the eyelashes in good condition and/or to improve their condition and/or appearance.

This invention also features administration of at least one 3-sulfanylpropanamide compound of formula (I) or a salt and/or solvate thereof, as an inhibitor of 15-hydroxyprostaglandin dehydrogenase, especially of type 1. The present invention also features the formulation of at least one 3-sulfanylpropanamide compound of formula (I) or a salt and/or solvate thereof, into a composition for treating disorders associated with the activity of type-1 15-hydroxyprostaglandin dehydrogenase, in particular in man.

The present invention also features a cosmetic regime or regimen for treating human keratin fibers (especially the hair or the eyelashes) and/or the skin, in particular that from which the said fibers emerge, for instance the scalp and the eyelids, comprising topically applying to the said keratin fibers and/or the said skin a cosmetic composition which comprises at least one compound of formula (I) or a salt and/or solvate thereof, leaving this composition in contact with the said keratin fibers and/or the said skin, and optionally rinsing the said fibers and/or the said skin.

This treatment does indeed have the characteristics of a cosmetic regime or regimen insofar as it makes it possible to improve the aesthetic appearance of human keratin fibers and especially the hair and the eyelashes and also the aesthetic appearance of the skin by imparting to them greater vigor and/or an improved appearance. In addition, it may be used daily for several months, without medical prescription.

More especially, the present invention features a cosmetic regime or regimen for caring for human hair and/or the scalp, to improve their condition and/or their appearance, comprising topically applying to the hair and/or to the scalp a cosmetic composition containing an effective amount of at least one compound of formula (I) or a salt and/or solvate thereof, leaving the composition in contact with the hair and/or the scalp, and optionally rinsing the hair and/or the scalp.

This invention also features a cosmetic regime or regimen for caring for human skin, in order to improve its condition and/or its appearance, comprising topically applying to the said skin a cosmetic composition containing an effective amount of at least one compound of formula (I) or a salt and/or solvate thereof, leaving the composition in contact with the said skin, and optionally rinsing it off.

This invention also features a cosmetic regime or regimen for caring for and/or making up human eyelashes, to improve their condition and/or their appearance, comprising topically applying to the eyelashes and/or the eyelids a mascara composition containing at least one compound of formula (I) or a salt and/or solvate thereof, and then leaving the composition in contact with the eyelashes and/or the eyelids. This mascara composition may be applied alone or as a base coat for a standard pigmented mascara and may be removed like a standard pigmented mascara.

The present invention also features the cosmetic administration of a 3-sulfanylpropanamide compound of formula (I) or a salt and/or solvate thereof, as an agent for preserving the amount and/or the activity of the prostaglandins, especially in the hair follicles.

This invention also features the formulation of at least one 3-sulfanylpropanamide compound of formula (I) or a salt and/or solvate thereof, into a composition for preserving the amount and/or the activity of the prostaglandins especially in the hair follicles.

Among the compounds of formula (I), a certain number of these are novel and constitute another aspect of the present invention.

These are the 3-sulfanylpropanamide compounds that satisfy at least one of the formulae (III), (IV), (V), (VII) and (VIII) below or one of the corresponding salts and/or solvates thereof: 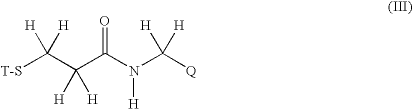
in which Q is:
a) a phenyl radical substituted with at least one radical Z; or
b) an aromatic heterocycle selected from the group consisting of pyrimidine, quinoline, isoquinoline, pyrrole, furan and thiophene, optionally substituted with at least one radical Z,
wherein Z represents:
   fluorine,
   the groups CF₃, CN, OR, SR, NRR′, SO₂NRR′, NRSO₂R′, SO₂R and SiRR′R″, wherein R, R′ and R″ have the same definitions as above,
   a group of formula (II): 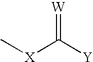
wherein W, X and Y have the same definitions as above;
   a C₁-C₁₀ alkyl radical optionally substituted with at least one substituent A₄, wherein A₄ has the same definition as above; and wherein T is:
a) a saturated alkyl radical selected from among methyl, ethyl, n-butyl, isobutyl and tert-butyl; or
b) a saturated C₄-C₆ hydrocarbon-based ring:

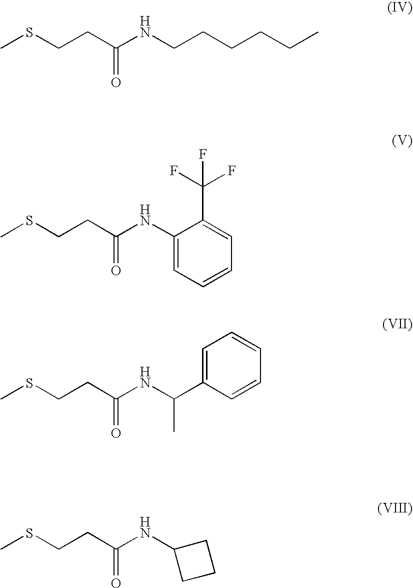

The present invention also features care and/or mascara compositions for the hair or the eyelashes, containing a cosmetically acceptable medium for topical application and a 3-sulfanylpropanamide compound that satisfies at least one of the formulae (III), (IV), (V), (VII) and (VIII) below, or a salt and/or solvate thereof.

This invention also features care and/or makeup compositions for keratin fibers, comprising, formulated into a physiologically acceptable medium, in particular a cosmetic medium, at least one 3-sulfanylpropanamide compound that satisfies at least one of the formulae (III), (IV), (V), (VII) and (VIII) below or a salt and/or solvate thereof and at least one additional active agent that promotes the regrowth of human keratin fibers and/or that limits their loss, selected from the group consisting of aminexil, FP receptor agonists, prostaglandins and derivatives thereof, and vasodilators, and more especially selected from among aminexil, minoxid i, latanoprost, (5E)-7-{(1R,2R,3R,5S)-3,5-dihydroxy-2-[(3R)-3-hydroxy-5-phenylpentyl]cyclopentyl}hept-5-enoic acid, butaprost, travoprost, diethyl 2,4-pyridinedicarboxylate and 6-0-[(9Z,12Z)octadeca-9,12-dienoyl]hexapyranose, and mixtures thereof.

The term “15-hydroxyprostaglandin dehydrogenase inhibitor” means a compound of formula (I) capable of inhibiting or reducing the activity of the enzyme 15-PGDH, especially of type 1 and in particular human 15-PGDH, and/or capable of inhibiting, reducing or slowing down the reaction catalyzed by this enzyme.

According to one advantageous embodiment of the invention, the compound of formula (I) is a specific inhibitor of 15-PGDH, in particular of type 1; the term “specific inhibitor” means an active agent that has little or no inhibitory effect on prostaglandin synthesis, in particular on the synthesis of PGF2-α or PGE2. According to one particular embodiment of the invention, the inhibitor of 15-PGDH especially of type 1 has little or no inhibitory effect on prostaglandin synthase (abbreviated to PGF synthase or PGFS).

Specifically, the assignee hereof has found that PGF synthase is also expressed in the dermal papilla. Maintenance of an effective amount of prostaglandins at the site of action thus results from a complex biological equilibrium from the synthesis and the degradation of these molecules. The exogenous supply of compounds that inhibit catabolism will thus be less effective if this activity is combined with an inhibition of the synthesis of these prostaglandins.

Advantageously, the compounds of formula (I), in salified or non-salified, solvated or non-solvated form, have inhibitory activity on 15-PGDH that is higher than the inhibitory activity on PGF synthase, and are thus selective inhibitors. In particular, the ratio from the inhibitory activities on PGF synthase and on 15-PGDH, respectively, for a given concentration, determined especially by the concentrations that inhibit 50% of the enzymatic activity of PGF synthase (IC_{50fc}) relative to the concentration that inhibits 50% of the enzymatic activity of 15-PGDH (IC_{50dh}) is at least greater than 1 and especially at least 3:1.

In the text hereinbelow, and unless otherwise mentioned, the use of the term “compound of formula (I)” should be understood as meaning either the compound of formula (I) or a salt or solvate and in particular a hydrate thereof, or a solvated salt (in particular a hydrated salt) thereof.

According to the invention, the compounds of formula (I) are in isolated form, i.e., non-polymeric form.

According to the invention, the term “salts of a compound of formula (I)” means the organic or mineral salts of a compound of formula (I).

As mineral salts according to the invention, exemplary are the sodium or potassium salts and also the ammonium, zinc (Zn²⁺), calcium (Ca²⁺), copper (Cu²⁺), iron (Fe²⁺ and Fe³⁺), strontium (Sr²⁺), magnesium (Mg²⁺) and manganese (Mn²⁺) salts; hydroxides, hydrohalides (for example hydrochlorides), carbonates, hydrogen carbonates, sulfates, hydrogen phosphates, phosphates.

The organic salts according to the invention are, for example, the triethanolamine, monoethanolamine, diethanolamine, hexadecylamine, N,N,N′,N′-tetrakis(2-hydroxypropyl)ethylenediamine, tris-hydroxymethylaminomethane, hydroxide, hydrohalide, carbonate, hydrogen carbonate, citrate, lactate, glycolate, gluconate, acetate, propionate, fumarate, oxalate, tartrate, sulfate, phosphate and hydrogen phosphate salts.

As solvates of the compounds of formula (I), exemplary are the hydrates, alcoholates and hydroalcoholates.

According to the invention, the term “at least one” means one or more (2, 3 or more). In particular, the composition may contain one or more compounds of formula (I). This or these compound(s) may be cis or trans or Z or E isomers or a mixture of cis/trans or Z/E isomers. They may also be in tautomeric form. This or these compound(s) may be enantiomers and/or diastereoisomers or a mixture of these isomers, in particular a racemic mixture.

For the purposes of the invention, the term “hydrocarbon-based” means a group of hydrogen and carbon atoms.

For the purposes of the invention, the term “alkyl radical” means a hydrocarbon-based radical that may be linear or branched and saturated or unsaturated. In particular, the alkyl radical contains from 1 to 20 and preferably from 1 to 10 carbon atoms. Exemplary alkyl radicals are the methyl, ethyl, isopropyl, n-butyl, tert-butyl, n-hexyl, 2-ethylhexyl, ethylene and propylene radicals.

According to the invention, the rings C¹ to C⁷ and Hy₂ of formula (I) contain from 3 to 7 atoms, for example 4, 5 or 6 atoms, and may be saturated or unsaturated, unless otherwise indicated. In addition, the heterocycles may contain from 1 to 4 hetero atoms, which may be identical or different, selected from N, S and O and a combination thereof. These rings may moreover be fused to another ring of identical or different chemical nature, to form fused rings. In addition, these rings may be substituted with one or more identical or different substituents, in particular A₂ or A₄. These rings may be linked via any atom thereof capable of forming a covalent bond with a carbon atom.

Exemplary saturated hydrocarbon-based rings according to the invention are cyclopropyl, cyclobutyl, cyclopentyl, cyclohexyl and cycloheptyl radicals. Unsaturated hydrocarbon-based rings include cyclohexenyl and phenyl rings. As fused hydrocarbon-based rings, exemplary are aryl radicals, for instance the naphthyl ring. Unsaturated hydrocarbon-based rings that are preferred are aryl rings, for instance phenyl or naphthyl rings.

Exemplary heterocycles according to the invention, in particular for the heterocycle Hy₂, are the following monoheterocycles: azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, dihydrothiazole, thiazole, dihydrothiazole, thiazolidine, pyrazole, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine, diazepine. Preferably, Hy₂ is a pyrrole, furan, dihydrofuran, tetrahydrofuran, thiophene, thiazole, oxazole, oxazolidine, isoxazole, isoxazolidine, oxadiazole, pyridine, pyran, dihydropyran, tetrahydropyran, pyrimidine, piperazine, pyridazine, pyrazine, morpholine, quinoline, isoquinoline, 2-thiohydantoin, 2-oxo-2,3-dihydro-1,3-thiazole or pyridone ring.

This monoheterocycle Hy₂ may also be fused to a saturated or unsaturated hydrocarbon-based or heterocyclic ring C⁷ of 4 to 7 atoms and better still of 5 to 6 atoms, which may be identical to or different from the heterocycle Hy₂. When this fused ring C⁷ is a heterocycle, it may contain from 1 to 4 identical or different hetero atoms selected from among O, N and S and a combination thereof.

Exemplary fused heterocycles according to the invention are purine and pteridine rings. Exemplary heterocycles fused to a hydrocarbon-based ring according to the invention include benzofuran, benzothiophene, benzothiazole, indole, benzimidazole, quinoline, isoquinoline and quinazoline radicals.

Advantageously, one from among R₁ and R₂ is hydrogen.

In particular, the 3-sulfanylpropanamide derivatives have the formula (la) below, or a corresponding salt and/or solvate thereof: 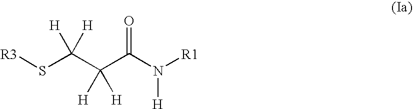
in which R₁ and R₃ have the same definitions as above.

Preferably, R₁ is a linear or branched, saturated or unsaturated C₁ to C₁₀ alkyl radical or a saturated or unsaturated hydrocarbon-based ring, this radical or this ring being substituted with an unsaturated hydrocarbon-based or heterocyclic ring C⁵, this ring C⁵ itself being optionally substituted with one or more substituents A₁, which may be identical or different, selected in particular from:
linear or branched, saturated or unsaturated C₁ to C₁₀ alkyl radicals, preferably saturated C₁-C₆ alkyl radicals and more preferentially methyl and ethyl radicals;
C₁-C₆ alkoxy radicals, preferentially methoxy;
aryl rings such as phenyl and naphthyl.

Preferably, the (hetero)ring C⁵ is furan or phenyl. Advantageously, this ring C⁵ is a phenyl radical, optionally substituted with a trifluoromethyl group.

According to one embodiment of the invention, R³ is a linear or branched, saturated or unsaturated C₁ to C₁₀ alkyl radical or a saturated or unsaturated hydrocarbon-based ring. More preferentially, R₃ is selected from among methyl, tert-butyl, cyclopentyl and cyclohexyl.

Exemplary 3-sulfanylpropanamide compounds of formula (I) and more particularly of formula (Ia) according to the invention include the following compounds:
3-(tert-butylsulfanyl)-N-[(5-methyl-2-furyl)methyl]propanamide,
N-cyclobutyl-3-(methylsulfanyl)propanamide,
3-(methylsulfanyl)-N-(1-phenylethyl)propanamide,
N-benzyl-3-(methylsulfanyl)propanamide,
3-(methylsulfanyl)-N-[2-(trifluoromethyl)phenyl]propanamide,
N-(4-methoxybenzyl)-3-(methylsulfanyl)propanamide,
N-(2-furylmethyl)-3-(methylsulfanyl)propanamide;
   and in particular: N-[(5-methyl-2-furyl)methyl]-3-methylsulfanyl)propanamide.

According to another embodiment of the invention, novel compounds are provided corresponding to formula (I) and satisfying one of the formulae (III), (IV), (V), (VII) and (VIII) below or a corresponding salt and/or solvate thereof: 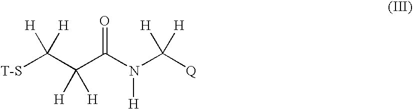
in which Q is:
a) a phenyl radical substituted with at least one radical Z; or
b) an aromatic heterocycle selected from among pyrimidine, quinoline, isoquinoline, pyrrole, furan and thiophene, optionally substituted with at least one radical Z,
wherein Z represents:
   fluorine,
   the groups CF₃, CN, OR, SR, NRR′, SO₂NRR′, NRSO₂R′, SO₂R or SiRR′R″,
wherein R and R′ have the same definitions as above,
   a group of formula (II): 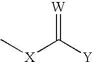
      wherein: W=O, NH, S
   X=O, NH, C₁-C₆ N-alkyl
   Y=C₁-C₄ alkyl, C₁-C₆ alkoxy, NH₂, C₁-C₆ NH-alkyl, C₁-C₆ N-dialkyl
   a C₁-C₁₀ alkyl radical optionally substituted with at least one substituent A₄;
      and in which T is:
   a) a saturated alkyl radical selected from among methyl, ethyl, n-butyl, isobutyl and tert-butyl; or
   b) a saturated C₄-C₆ hydrocarbon-based ring;

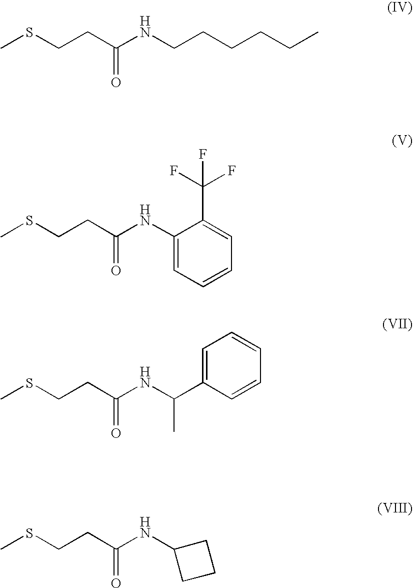

Exemplary compounds according to the invention include the following:

Compound 1: N-[(5-methyl-2-furyl)methyl]-3-(methylsulfanyl)propanamide:

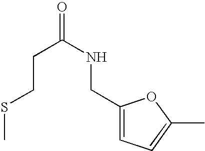

Compound 2: N-(2-furylmethyl)-3-(methylsulfanyl)propanamide:

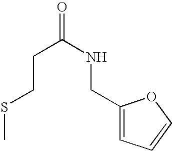

Compound 3: N-(4-methoxybenzyl)-3-(methylsulfanyl)propanamide:

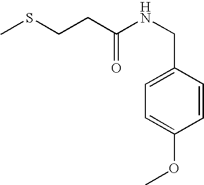

Compound 4: N-hexyl-3-(methylsulfanyl)propanamide:

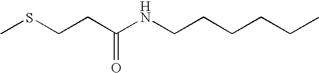

Compound 5: 3-(methylsulfanyl)-N-[2-(trifluoromethyl)phenyl]propanamide:

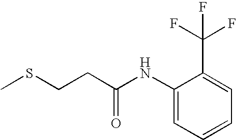

Compound 6: N-benzyl-3-(methylsulfanyl)propanamide:

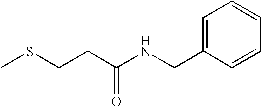

Compound 7: 3-(methylsulfanyl)-N-(1-phenylethyl)propanamide:

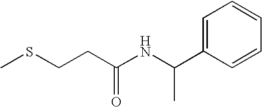

Compound 8: N-cyclobutyl-3-(methylsulfanyl)propanamide:

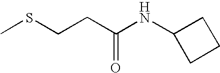

Compound 9: 3-(tert-butylsulfanyl)-N-[(5-methyl-2-furyl)methyl]propanamide:

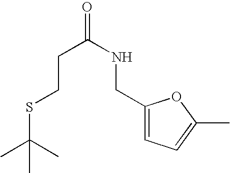

Compound 10: 3-(tert-butylsulfanyl)-N-(2-furylmethyl)propanamide:

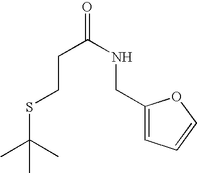

Compound 11: 3-(cyclopentylsulfanyl)-N-[(5-methyl-2-furyl)methyl]propanamide:

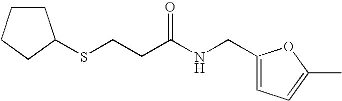

Compound 12: 3-(cyclopentylsulfanyl)-N-(2-furylmethyl)propanamide:

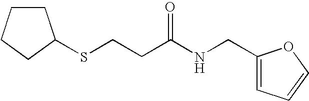

Compound 13: 3-(tert-butylsulfanyl)-N-[2-(trifluoromethyl)phenyl]propanamide:

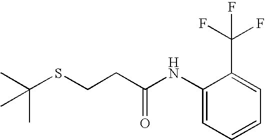

Compound 14: 3-[(3,3,4,4,5,5,6,6,7,7,8,8,9,9,10,10,10-heptadecafluorodecyl)sulfanyl]-N-{4-[(Z)-(4,7,7-trimethyl-3-oxobicyclo[2.2.1]hept-2-ylidene)methyl]benzyl}propanamide:

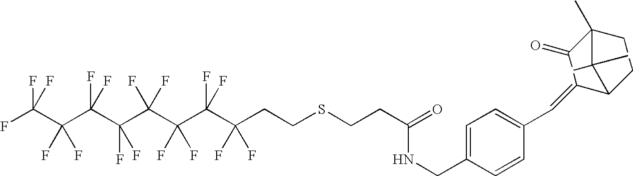

Compound 15: N-[3-(2H-1,2,3-benzotriazol-2-yl)-2-hydroxy-5-(1,1,3,3-tetramethylbutyl)benzyl]-3-[(3,3,4,4,5,5,6,6,7,7,8,8,9,9,10,10,10-heptadecafluorodecyl)sulfanyl]propanamide:

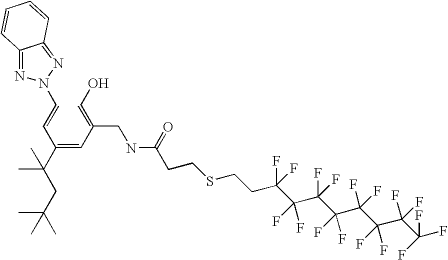

Compound 16: 3-[(3,3,4,4,5,5,6,6,7,7,8,8,8-tridecafluorooctyl)sulfanyl]-N-{4-[(Z)-(4,7,7-trimethyl-3-oxobicyclo[2.2.1]hept-2-ylidene)methyl]benzyl}propanamide:

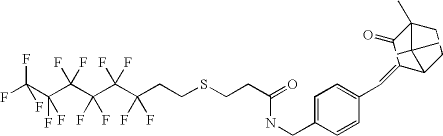

Exemplary novel compounds according to the invention that satisfy one of the formulae (III), (IV), (V), (VII) and (VIII) include the compounds 1, 2, 3, 4, 5, 7 and 8.

The salified or non-salified and/or solvated or non-solvated compounds of formula (I) may be prepared according to the reaction scheme below:

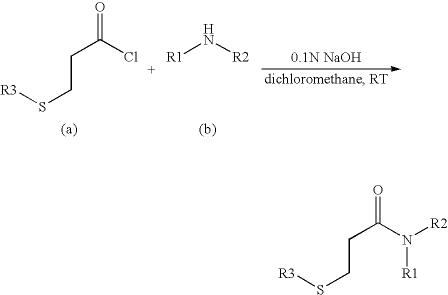

One equivalent of product (a) is mixed with 1.1 equivalents of amine (b) as solution in a dichloromethane/aqueous sodium hydroxide two-phase mixture. The reaction is carried out for 4 hours at room temperature. The reaction medium is then left to settle. The dichloromethane phase is separated out and then dried over magnesium sulfate. The solvent is then evaporated off to give the desired compound in crystalline form.

The acid chlorides (a) are either commercially available or are prepared in two steps on the basis of the following publications:

Step 1: 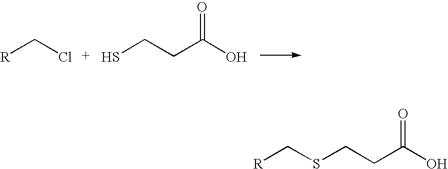
for example: 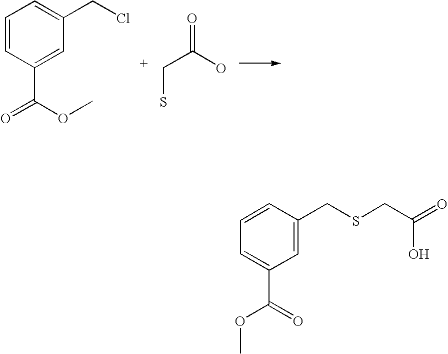
“Synthesis of Some Novel α,β-Ethylenic Sulfones,” Reddy M. V. R., Vijayalakshmi S., Reddy D. B., Reddy P. V. R., Phosphorus, Sulfur Silicon Relat. Elem., 60 (1991) 3-4, 209-214;
or alternatively: 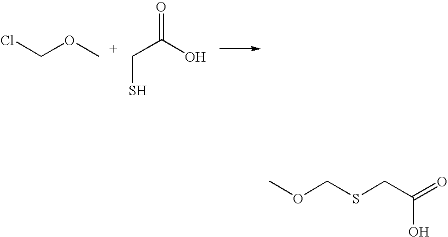
“Selective Preparation of β-Monoesters of Mercaptosuccinic Acid with Acid or Base Labile Sulfur Protecting Groups and Esters,” Gustavson L. M., Jones D. S., Nelson J. S., Srinivasan, A., Synth. Commun., 21 (1991) 2, 249-263;
or alternatively: 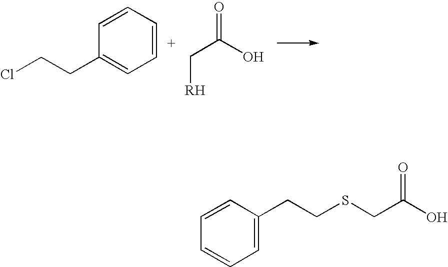
“A Novel and Convenient Synthesis of E,E,-Bis(styryl)sulfones and E,E-1,4-Diaryl-1,3-butadienes,” Reddy M. V. R., Manjubhashini A. B., Reddy S., Reddy P. V. R., Reddy D. B., Synth. Commun., 21 (1991) 15-16, 1589-1596.

Step 2: 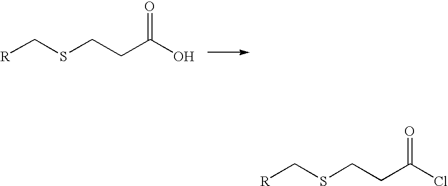
for example: 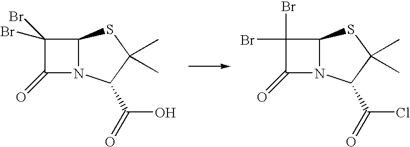
“Simple and Convenient Procedure for the Preparation of Acid Chlorides of Penicillins,” Mata E. G., Boschetti C. E., Mascaretti O. A., Org. Prep. Proced. Int., 27 (1995) 2, 229-232.

To the assignee's knowledge, no prior art document describes or suggests that the 3-sulfanylpropanamide compounds of formula (I) or salts and/or solvates thereof have the property of inducing and/or stimulating the growth of human keratin fibers and in particular human hair and eyelashes, and/or of stopping their loss and/or of stimulating the pigmentation of the skin and/or keratin fibers and/or of limiting their pigmentation and/or bleaching, or that these compounds may be used topically to increase the density of human keratin fibers and/or to limit their bleaching and more especially to increase the density and/or bleaching of the hair and the eyelashes.

The effective amount of a compound of formula (I) (salified or non-salified, and solvated or non-solvated) corresponds to the amount of compound required to obtain the desired result (i.e., to increase the density of keratin fibers and especially the hair and the eyelashes or to promote their growth or reduce their bleaching). One skilled in the art is thus capable of evaluating this effective amount, which depends on the nature of the compound used, the person on whom it is applied and the time of this application.

In the text hereinbelow, and unless otherwise indicated, the amounts of the various ingredients in the composition are given as weight percentages relative to the total weight of the composition.

To provide an order of magnitude, according to the invention, the compound of formula (I) (salified or non-salified, and solvated or non-solvated) or a mixture of compounds of formula (I) (salified or non-salified, and solvated or non-solvated) may be present in an amount ranging from 10⁻³% to 10% of the total weight of the composition, preferably in an amount representing from 10⁻³% to 5% and better still from 10⁻² to 2% of the total weight of the composition, for example from 0.5% to 2%.

The compositions of the invention may be for cosmetic or pharmaceutical applications. The compositions of the invention are preferably for cosmetic applications. In addition, the compositions must contain a non-toxic, physiologically acceptable medium that can be topically applied to human skin, including the scalp and the eyelids and to keratin fibers. For the purposes of the invention, the term “cosmetic” means a composition of pleasant appearance, odor and feel.

The compound of formula (I) (salified or non-salified, and solvated or non-solvated) may be formulated into compositions that should be ingested, injected or applied to the skin or to keratin fibers (to any area of skin or fibers to be treated).

According to the invention, the compound of formula (I) or a mixture of compounds of formula (I) may be administered orally in an amount of from 0.1 to 300 mg per day, for example from 5 to 10 mg/day.

A preferred composition of the invention is a composition for cosmetic application and in particular for topical application to the skin and keratin fibers, and more especially to the scalp, the hair and the eyelashes.

This composition may be in any known presentation form that is suitable for the mode of use.

For topical application to the skin or keratin fibers, the composition may be in the form of an aqueous, alcoholic or aqueous-alcoholic solution or suspension, or an oily suspension or solution, an emulsion or dispersion of more or less fluid consistency and especially of liquid or semi-liquid consistency, obtained by dispersion of a fatty phase in an aqueous phase (O/W) or conversely (W/O), a solid (O/W) or (W/O) emulsion or dispersion, a more or less fluid or solid aqueous, aqueous-alcoholic or oily gel, a free or compacted powder to be used in unmodified form or to be incorporated into a physiologically acceptable medium, or alternatively microcapsules, microparticles or vesicular dispersions of ionic and/or nonionic type.

A composition in the form of a foam or alternatively in the form of a spray or aerosol, then comprising a pressurized propellant, is also intended.

It may also be in the form of a lotion, serum, milk, O/w or W/O cream, gel, unguent, ointment, powder, balm, patch, impregnated pad, cake or foam.

In particular, the composition for application to the scalp or the hair may be in the form of a haircare lotion, for example for daily or twice-weekly application, a shampoo or a hair conditioner, in particular for twice-weekly or weekly application, a liquid or solid scalp cleansing soap for daily application, a hairstyle shaping product (lacquer, hair setting product or styling gel), a treatment mask, a foaming gel or cream for cleansing the hair. It may also be in the form of a hair dye or mascara to be applied with a brush or a comb.

Moreover, for application to the eyelashes or body hairs, the composition of the invention may be in the form of a pigmented or unpigmented mascara, to be applied with a brush to the eyelashes or alternatively to beard or moustache hair.

For a composition administered by injection, it may be in the form of an aqueous lotion or an oily suspension. For oral administration, the composition may be in the form of capsules, granules, drinkable syrups or tablets.

According to one particular embodiment, the composition according to the invention is in the form of a hair cream or hair lotion, a shampoo, a conditioner for the hair or a mascara for the hair or for the eyelashes.

The amounts of the various constituents of the physiological medium of the compositions according to the invention are those generally employed in the fields under consideration. In addition, these compositions are prepared according to the usual methods.

When the composition is an emulsion, the proportion of the fatty phase may range from 2% to 80% by weight and preferably from 5% to 50% by weight relative to the total weight of the composition. The aqueous phase is adjusted as a function of the content of fatty phase and of compound(s) (I) and also of that of the optional additional ingredients, to obtain 100% by weight. In practice, the aqueous phase is from 5% to 99.9% by weight.

The fatty phase may contain fatty or oily compounds that are liquid at room temperature (25° C.) and atmospheric pressure (760 mm Hg), which are generally known as oils. These oils may be mutually compatible or incompatible and may form a macroscopically homogeneous liquid fatty phase or a two-phase or three-phase system.

In addition to the oils, the fatty phase may contain waxes, gums, lipophilic polymers or “pasty” or viscous products containing solid parts and liquid parts.

The aqueous phase contains water and optionally an ingredient that is miscible in all proportions with water, for instance C₁ to C₈ lower alcohols such as ethanol or isopropanol, polyols, for instance propylene glycol, glycerol or sorbitol, or alternatively acetone or ether.

For a composition in emulsion form, the composition may contain one or more emulsifiers optionally combined with one or more co-emulsifiers used to obtain a composition in emulsion form, these emulsifiers and co-emulsifiers being those generally used in cosmetics and pharmaceuticals. Their nature also depends on the sense of the emulsion. In practice, the emulsifier and, where appropriate, the co-emulsifier are present in the composition in a proportion ranging from 0.1% to 30% by weight, preferably from 0.5% to 20% by weight and better still from 1% to 8% by weight. The emulsion may also contain microcapsules or microparticles, and vesicular dispersions and especially lipid vesicles and especially liposomes.

When the composition is in the form of an oily solution or gel, the fatty phase may represent more than 90% of the total weight of the composition.

Advantageously, for a hair application, the composition of the invention is an aqueous, alcoholic or aqueous-alcoholic solution or suspension and better still a water/ethanol solution or suspension. The alcoholic fraction may represent from 5% to 99.9% and better still from 8% to 80%.

For a mascara application, the composition of the invention is especially in the form of a wax-in-water or wax-in-oil dispersion, a gelled oil or an aqueous gel, which may be pigmented or unpigmented.

The compositions of the invention may also comprise other additional ingredients usually employed in the fields under consideration, selected from among solvents, aqueous-phase or oily-phase solvents, thickeners or gelling agents, dyes that are soluble in the medium of the composition, solid particles such as fillers or pigments, antioxidants, sequestrants, preservatives, fragrances, electrolytes, neutralizers, film-forming polymers, UV blockers, for instance sunscreens, cosmetic and pharmaceutical active agents with a beneficial effect on the skin or keratin fibers, other than the compounds of formula (I), and mixtures thereof. These additives may be present in the composition in the amounts generally employed in cosmetics and dermatology, and especially in a proportion of from 0.01% to 50% and better still from 0.1% to 20%, for example from 0.1% to 10%, relative to the total weight of the composition. Depending on their nature, these additives may be introduced into the fatty phase, into the aqueous phase and/or into the lipid vesicles and especially liposomes.

Needless to say, one skilled in the art will take care to select the optional additional ingredients and/or the amount thereof such that the advantageous properties of the composition according to the invention, i.e., the inhibition of 15-PGDH and in particular the increase in the density of keratin fibers and/or the reduction in their bleaching, are not, or are not substantially, adversely affected by the envisaged addition.

As solvents according to the invention, exemplary are C₂ to C₈ lower alcohols, for instance ethanol, isopropanol, propylene glycol and certain light cosmetic oils, for instance C₆ to C₁₆ alkanes.

As oils according to the invention, exemplary are oils of mineral origin (liquid petroleum jelly or hydrogenated isoparaffin), oils of plant origin (liquid fraction of shea butter, sunflower oil, apricot oil, fatty alcohol or fatty acid), oils of animal origin (perhydrosqualene), synthetic oils (fatty acid ester, purcellin oil), silicone oils (linear or cyclic polydimethylsiloxane, phenyl trimethicone) and fluoro oils (perfluoropolyethers). Waxes that are exemplary include silicone waxes, beeswax, rice wax, candelilla wax, carnauba wax, paraffin wax and polyethylene wax.

As emulsifiers according to the invention, exemplary are glyceryl stearate, glyceryl laurate, sorbitol stearate, sorbitol oleate, alkyl dimethicone copolyols (with alkyl ≧8) and mixtures thereof for a W/O emulsion. Polyethylene glycol monostearate or monolaurate, polyoxyethylenated sorbitol stearate or oleate, and dimethicone copolyols, and mixtures thereof, may also be employed for an ONV emulsion.

As hydrophilic gelling agents according to the invention, exemplary are carboxyvinyl polymers (carbomer), acrylic copolymers such as acrylate/alkylacrylate copolymers, polyacrylamides, polysaccharides such as hydroxypropylcellulose, natural gums and clays, and, as lipophilic gelling agents, exemplary are modified clays, for instance Bentones, metal salts of fatty acids, for instance aluminum stearates, hydrophobic-treated silica and ethylcellulose, and mixtures thereof.

As cosmetic or pharmaceutical active agents other than the compound of formula (I), which may be included in the invention, exemplary are hydrophilic active agents selected from among proteins or protein hydrolysates, amino acids, polyols, urea, allantoin, sugars and sugar derivatives, water-soluble vitamins, plant extracts (those from Iridacea plants or from soybean) and hydroxy acids such as fruit acids or salicylic acid; and lipophilic active agents such as retinol (vitamin A) and its derivatives, especially an ester (retinyl palmitate), tocopherol (vitamin E) and its derivatives, especially an ester (tocopheryl acetate), essential fatty acids, ceramides, essential oils, salicylic acid derivatives, for instance 5-n-octanoylsalicylic acid, hydroxy acid esters, and phospholipids, for instance lecithin, and mixtures thereof.

According to one particular embodiment of the invention, the compound of formula (I) may be combined with at least one additional active compound that promotes the regrowth and/or limits the loss of keratin fibers (hair or eyelashes). These additional compounds are selected especially from among the lipoxygenase inhibitors as described in EP-0,648,488, the bradykinin inhibitors described especially in EP-0,845,700, prostaglandins and derivatives thereof, especially those described in WO 98/33497, WO 95/11003, JP 97-100 091 and JP 96-134 242, prostaglandin receptor agonists or antagonists, the non-prostanoic prostaglandin analogues as described in EP-1,175,891, EP-1,175,890, WO 01/74307, WO 01/74313, WO 01/74314, WO 01/74315 or WO 01/72268, and mixtures thereof.

As other additional active compounds that promote the growth of keratin fibers and/or limit their loss, which may be present in the compositions according to the invention, exemplary are vasodilators, anti-androgens, cyclosporins and analogues thereof, anti-microbial and anti-fungal agents, anti-inflammatory agents, and retinoids, alone or in a mixture.

The vasodilators that are exemplary are especially potassium-channel agonists, including minoxidil, and also the compounds described in U.S. Pat. Nos. 3,382,247, 5,756,092, 5,772,990, 5,760,043, 5,466,694, 5,438,058 and 4,973,474, cromakalim, nicorandil and diaxozide, alone or in combination.

The anti-androgens that are exemplary especially include steroidal and non-steroidal 5α-reductase inhibitors, for instance finasteride and the compounds described in U.S. Pat. No. 5,516,779, cyprosterone acetate, azelaic acid and the salts and derivatives thereof, and the compounds described in U.S. Pat. No. 5,480,913, flutamide, oxendolone, spironolactone, diethylstilbestrol and the compounds described in U.S. Pat. Nos. 5,411,981, 5,565,467 and 4,910,226.

The anti-microbial or anti-fungal compounds may be selected from among selenium derivatives, octopirox, triclocarban, triclosan, zinc pyrithione, itraconazole, asiatic acid, hinokitiol, mipirocine, tetracyclines, especially erythromycin and the compounds described in EP-0,680,745, clinycin hydrochloride, benzoyl peroxide or benzyl peroxide, minocycline and compounds belonging to the imidazole class, such as econazole, ketoconazole or miconazole or salts thereof, nicotinic acid esters, especially including tocopheryl nicotinate, benzyl nicotinate and C₁-C₆ alkyl nicotinates, for instance methyl nicotinate or hexyl nicotinate.

The anti-inflammatory agents may be selected from among steroidal anti-inflammatory agents, for instance glucocorticoids, corticosteroids (for example: hydrocortisone) and non-steroidal anti-inflammatory agents, for instance glycyrrhetinic acid and α-bisabolol, benzydamine, salicylic acid and the compounds described in EP-0,770,399, WO 94/06434 and FR-2,268,523.

The retinoids may be selected from among isotretinoin, acitretin, tazarotene, retinal and adapalene.

As other active compounds for promoting the growth and/or limiting the loss of hair that may be used in combination with the compound of formula (I), which may or may not be salified and may or may not be solvated, exemplary are aminexil, diethyl 2,4-pyridinedicarboxylate, 6-0-[(9Z,12Z)octadeca-9,12-dienoyl]hexapyranose, benzalkonium chloride, benzethonium chloride, phenol, oestradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharide or acylhexosaccharide acids, substituted arylethylenes, N-acylamino acids, flavonoids, ascomycin derivatives and analogues, histamine antagonists, saponins, proteoglycanase inhibitors, oestrogen agonists and antagonists, pseudoterines, cytokines, growth factor promoters, IL-1 or IL-6 inhibitors, IL-10 promoters, TNF inhibitors, benzophenones, hydantoin, retinoic acid; vitamins, for instance vitamin D, vitamin B12 analogs and pantothenol; triterpenes, for instance ursolic acid and the compounds described in U.S. Pat. Nos. 5,529,769, 5,468,888 and 5,631,282; anti-pruriginous agents, for instance thenaldine, trimeprazine or cyproheptadine; anti-parasitic agents, in particular metronidazole, crotamiton or pyrethroids; calcium antagonists, for instance cinnarizine, diltiazem, nimodipine, verapamil, alverine and nifedipine; hormones such as oestriol or its analogues, thyroxine and its salts, and progesterone; FP receptor (type-F prostaglandin receptor) agonists such as latanoprost, (5E)-7-{(1R,2R,3R,5S)-3,5-dihydroxy-2-[(3R)-3-hydroxy-5-phenylpentyl]cyclopentyl}hept-5-enoic acid, (5E)-7-{(1R,2R,3R,5S)-3,5-dihydroxy-2-[(3R)-3-hydroxy-5-phenylpentyl]cyclopentyl}hept-5-enoic acid, bimatoprost, travoprost or unoprostone, salts or esters thereof; mixtures thereof.

Advantageously, the compositions according to the invention comprise at least one 15-PGDH inhibitor as defined above and at least one prostaglandin or prostaglandin derivative, for instance the prostaglandins of series 2 especially including PGF2-α and PGE2 in salt or ester form (for example the isopropyl esters), derivatives thereof, for instance 16,16-dimethyl PGE2, 17-phenyl PGE2, 16,16-dimethyl PGF2-α, 17-phenyl PGF2-α, prostaglandins of series 1, for instance 11-deoxyprostaglandin E1, 1-deoxyprostaglandin E1 in salt or ester form, analogues thereof, especially latanoprost, fluprostenol, bimatoprost, cloprostenol, viprostol, butaprost, misoprostol, unoprostone, and the salts or esters thereof.

The subject compositions advantageously contain at least one non-prostanoic EP2 and/or EP4 receptor agonist as described especially in EP-1,175,892.

It is also intended that the composition comprising at least the compound of formula (I), salified or non-salified, solvated or non-solvated, may be encapsulated in particular in liposomal form, as described especially in WO 94/22468. Thus, the compound encapsulated may be delivered selectively to the hair follicle.

The compositions according to the invention may be topically applied to the areas of the skin to be treated and in particular to the alopecic areas of the scalp and the hair of an individual, or only to the white hairs, and optionally left in contact for several hours and optionally rinsed off.

The compositions containing an effective amount of a compound of formula (I), salified or non-salified, solvated or non-solvated, may, for example, be applied in the evening, kept in contact throughout the night and optionally shampooed out in the morning. These applications may be repeated daily for one or more months according to the individual.

Advantageously, in the regime or regimen according to the invention, from 5 μL and 500 μL of a solution or composition as defined above, comprising from 0.001% to 5% of 15-PGDH inhibitor, are topically applied to the areas of the scalp and/or the hair to be cared for or treated.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Example 1

### Synthesis of N-[(5-methyl-2-furyl)methyl]-3-(methylsulfanyl)propanamide (compound 1):

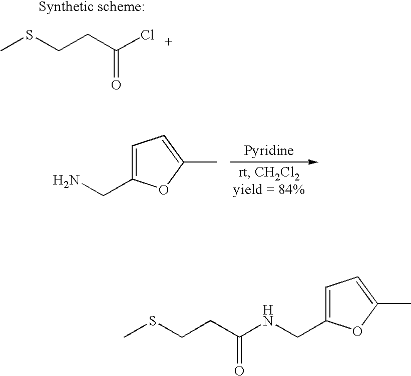

Procedure:

To a solution, cooled on an ice bath, of 3-methylthiopropionyl chloride (27.1 mL, 235 mM) in 500 mL of methylene chloride is added dropwise under nitrogen over 15 minutes a solution of 5-methylfurfurylamine (28 mL, 250 mM) in anhydrous pyridine (21 mL, 260 mM). The reaction medium is stirred for 24 hours. The reaction medium is then washed with aqueous 1 N hydrochloric acid solution (3 times 100 mL) and then with aqueous 1N sodium hydroxide solution, and then with water (3 times 100 mL). The organic phase is dried over sodium sulfate and the solvent is then evaporated off under vacuum. The product is washed with hot water. On cooling, the product solidifies. The solid obtained is finely divided and then stirred in 200 mL of water for 2 hours. The solid is then filtered off and dried to give 41.94 g (yield=84%) of a white powder.

Structural Analyses:

Nuclear magnetic resonance: The spectra obtained are in accordance with the proposed structure:

¹H NMR (DMSO-d6): 2.04 (s, 3H); 2.22 (d, 3H); 2.39 (t, 2H); 2.65 (t, 2H); 4.19 (d, 2H); 5.97 (dd, 1H); 6.09 (d, 1H); 8.27 (t, 1H).

Mass spectrometry: Analysis by ESI+ and ESI− makes it possible to confirm the molecular mass of the expected product C₁₀H₁₅NO₂S, M=213.

(Detection of [M+H]⁺, [M+Na]⁺, [M+H+CH₃OH]⁺, [2M+Na]⁺, [M−H]′ and of the fragment [M+H−C₄H₉H₉NOS]⁺).

### Examples 2, 9, 10, 11 and 12

Compounds 2, 9, 10, 11 and 12 may be synthesized via a synthetic method similar to that for compound 1.

### Example 2

### Structural Analyses

Nuclear magnetic resonance: The spectra obtained are in accordance with the proposed structure:

¹H NMR (DMSO-d6): 2.04 (s, 3H); 2.40 (t, 2H); 2.66 (t, 2H); 4.25 (d, 2H); 6.24 (m,1H); 6.38 (dd,1H); 7.56 (dd,1H); 8.27 (t, 1H).

Mass spectrometry: Analysis by ESI+ and ESI− makes it possible to confirm the molecular mass of the expected product C₉H₁₃NO₂S, M=199. (Detection of [M+H]⁺, [M+Na]⁺, [M+NaCH₃OH]⁺, [2M+H]⁺, [2M+Na]⁺, [M−H]⁻ and [M+CH₃COO]⁻).

### Example 3

### Synthesis of N-(4-methoxybenzyl)-3-(methylsulfanyl)propanamide (compound 3)

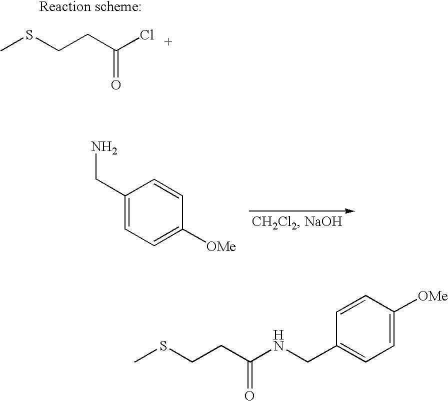

Procedure:

3-Methylthiopropionyl chloride (1 g, 7.25 mM) is added with stirring at room temperature to 4-methoxybenzylamine (2393-23-9) (8 mM, 1.1 g) dissolved in a dichloromethane (20 mL)/sodium hydroxide (0.8 M, 10 mL) mixture. The reaction is monitored for 1 hour and the mixture is then allowed to settle and the organic phase is washed with saturated aqueous sodium chloride solution. The organic phase is dried over sodium sulfate, filtered and concentrated under reduced pressure to give a crude oil (1.91 g). This oil is purified by chromatography on a column of silica gel, eluting with a 99/1 dichloromethane/methanol mixture to give compound 3 in the form of a white powder (yield=80%).

Analysis:

Elemental analysis:

| | | | | | |
|---|---|---|---|---|---|
| Calculated: | C 60.2; | H 7.2; | N 5.9; | O 13.4; | S 13.4 |
| Found: | C 60.15; | H 7.17; | N 5.81; | O 13.30; | S 13.22 |

Nuclear magnetic resonance: The spectra obtained are in accordance with the proposed structure:

¹H NMR (DMSO-d6): 2.05 (s, 3H); 2.41 (t, 2H); 2.67 (t, 2H); 3.72 (s, 3H); 4.2 (d, 2H); 6.87 (d, 2H); 7.18 (d, 2H); 8.28 (t, 1H).

### Example 4

### Synthesis of N-hexyl-3-(methylsulfanyl)propanamide (compound 4)

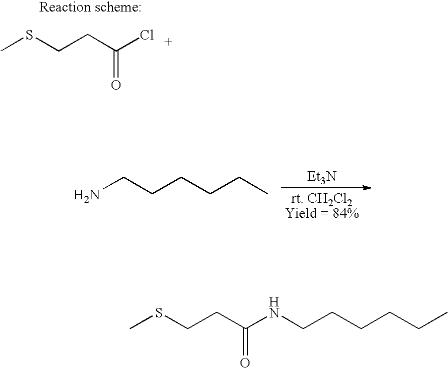

Procedure:

3-Methylthiopropionyl chloride (21 mL, 182 mM) is added with stirring at room temperature to a solution of hexylamine (26.5 mL, 200 mM) and triethylamine (28.1 mL, 200 mM) in dichloromethane. The reaction medium is stirred for two hours. The reaction mixture is then washed with hydrochloric acid (1N) and then washed with saturated aqueous sodium chloride solution. The organic phase is dried over sodium sulfate, filtered and concentrated under reduced pressure to give a colorless oil. This oil is distilled to give a colorless liquid that crystallizes under cold conditions (30.00 g; yield=81%).

Structural analyses:

Nuclear magnetic resonance: The spectra obtained are in accordance with the proposed structure:

¹H NMR (DMSO-d6): 0.86 (t, 3H); 1.25 (m, 6H); 1.38 (m, 2H); 2.04 (s, 3H); 2.33 (t, 2H); 2.63 (t, 2H); 3.02 (m, 2H); 7.8 (t, 1H).

Mass spectrometry: The quasimolecular ions [M+H]⁺, [M+Na]⁺, [M+Na+CH₃OH]⁺, [2M+H]⁺, [2M+Na]⁺, [M−H]⁻ and also the fragment ion [C₅H₁₁NO+H]+ (detected at m/z, ESP+=102) of the expected molecule C₁₀H₂₁NOS are mainly detected.

### Example 5

### Synthesis of 3-(methylsulfanyl)-N-[2-(trifluoromethyl)phenyl]propanamide (compound 5)

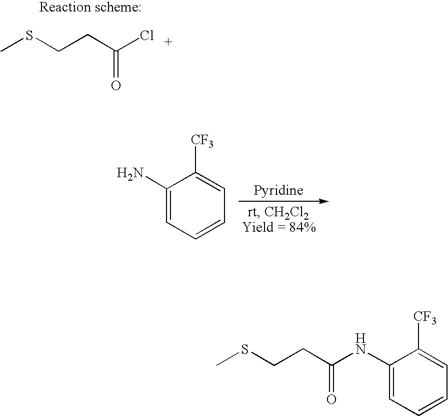

Procedure:

To a solution, cooled on an ice bath, of 3-methylthiopropionyl chloride (26 mL, 225 mM) in 500 mL of methylene chloride is added dropwise under nitrogen over 15 minutes a solution of 2-(trifluoromethyl)aniline (31 mL, 248 mM) in anhydrous triethylamine (35 mL, 284 mM). The reaction medium is stirred for 16 hours. The reaction medium is then washed with aqueous 1N hydrochloric acid solution (3 times 100 mL) and then with aqueous 1 N sodium hydroxide solution, and then with water (3 times 100 mL). The organic phase is dried over sodium sulfate and the solvent is then evaporated off under vacuum. The product obtained is distilled under vacuum to give an oil that solidifies under cold conditions to give 39.20 g of a beige-colored solid (yield=66%).

Structural Analyses:

Nuclear Magnetic Resonance: The spectra obtained are in accordance with the proposed structure:

¹H NMR (DMSO-d6): 2.1 (s, 3H); 2.64 (m, 2H); 2.73 (d, 1H); 2.75 (dd, 1H); 7.44 (d, 1H); 7.48 (m, 1H); 7.67 (m, 1H); 7.72 (d, 1H); 9.58 (s, 1H).

Mass spectrometry: The quasimolecular ions [M+H]⁺, [M+Na]⁺, [M+Na+CH₃OH]⁺, [2M+Na]⁺, [M−H]⁻ of the expected molecule C₁₁H₁₂F₃NOS are mainly detected.

### Examples 6, 7, 8 and 13

Compounds 6, 7, 8 and 13 may be synthesized via a synthetic method similar to that for compound 5.

### Example 7

### Structural Analyses:

Nuclear Magnetic Resonance: The spectra obtained are in accordance with the proposed structure:

¹H NMR (CDCl₃): 2.08 (s, 3H); 2.45 (t, 2H); 2.76 (t, 2H); 4.42 (d, 1H); 5.91 (se, 1H); 7.28 (m, 5H).

Mass spectrometry: Analysis by ESI+ and ESI− makes it possible to confirm the molecular mass of the expected product C₁₂H₁₇NOS, M=223. (Detection of [M+H]⁺, [M+Na]⁺, [2M+H]⁺, [2M+Na]⁺, [M−H]⁻.

### Example 8

### Structural Analyses

Nuclear Magnetic Resonance: The spectra obtained are in accordance with the proposed structure:

¹H NMR (DMSO-d6): 1.6 (m, 2H); 1.83 (m, 2H); 2.03 (s, 3H); 2.12 (m, 2H); 2.3 (t, 2H); 2.61 (t, 2H); 4.16 (m, 1H); 8.11 (d, 1H).

Mass spectrometry: Analysis by ESI+ and ESI− makes it possible to confirm the molecular mass of the expected product C₈H₁₅NOS, M=173. (Detection of [M+H]⁺, [M+Na]⁺).

### Examples 14 and 15

The synthesis of compounds 14 and 15 is that described in EP 652 210.

### Example 16

Compound 16 may be synthesized via a synthetic method similar to that for compound 14.

### Example 17

### Demonstration of the 15-PGDH-Specific Inhibitory Properties of the Compounds of Formula (I):

1) Test on 15-PGDH:

The enzyme 15-PGDH is obtained as described in FR 02/05067 filed in the name of L'Oreal, as a suspension in a medium adjusted to a concentration of 0.3 mg/mL and then blocked at −80° C. For the purposes of the test, this suspension is thawed and stored in ice.

In parallel, a 100 mM, pH 7.4 Tris buffer containing 0.1 mM of dithiothreitol (D5545, Sigma-Aldrich, L'isle D'Abeau Chesne, BP 701, 38297, Saint Quentin Fallavier), 1.5 mM of β-NAD (N6522, Sigma-Aldrich, L'isle D'Abeau Chesne, BP 701, 38297, Saint Quentin Fallavier), and 50 μM of prostaglandin E₂ (P4172, Sigma-Aldrich, L'isle D'Abeau Chesne, BP 701, 38297, Saint Quentin Fallavier) is prepared.

0.965 ml of this buffer (brought to 37° C. beforehand) is introduced into the cuvette of a spectrophotometer (Perkin-Elmer, Lambda 2) thermostatically maintained at 37° C., the measuring wavelength of which is set at 340 nm. 0.035 mL of enzymatic suspension at 37° C. is introduced into the cuvette concomitantly with the recording (corresponding to an increase in the optical density at 340 nm). The maximum reaction rate is recorded.

The test values (containing the compounds (I)) are compared with the control value (without compound (I)); the results indicated represent the concentration at which the compound of formula (I) reduces the enzymatic activity of 15-PGDH by 50%, namely IC_{50dh}.

2) Test on PGF Synthase:

The enzyme PGFS is obtained as described in FR-A-02/05067, at a concentration of 0.5 mg/mL, as a suspension in a suitable medium, and blocked at −80° C. For the purposes of the test, this suspension is thawed and stored in ice.

In parallel, a 100 mM, pH 6.5 Tris buffer containing 20 μM of 9,10-phenanthrenequinone* (P2896, Sigma-Aldrich, L'isle D'Abeau Chesne, BP 701, 38297, Saint Quentin Fallavier) and 100 μM of P-NADPH (N1630, Sigma-Aldrich, L'isle DAbeau Chesne, BP 701, 38297, Saint Quentin Fallavier) is prepared in a brown flask (protected from light). *A stock solution with a titre of 1 mM is prepared in absolute ethanol and brought to 40° C.; the flask is placed in an ultrasound cuvette to facilitate the dissolution of the product.

0.950 mL of this buffer (brought to 37° C. beforehand) is introduced into the cuvette of a spectrophotometer (Perkin-Elmer, Lambda 2) thermostatically maintained at 37° C., the measuring wavelength of which is set at 340 nm. 0.05 mL of enzymatic suspension at 37° C. is introduced into the cuvette concomitantly with the recording (corresponding to a reduction in the optical density at 340 nm). The maximum reaction rate is recorded.

The test values (containing compound (I)) are compared with the control value (without compound (I)); the results indicated represent the concentration at which the compound of formula (I) reduces the enzymatic activity of PGFS by 50%, namely IC_{50fs}.

The results are given in the table below:

| | | IC50 |
|---|---|---|
| Example | STRUCTURE | (μM) |
| 1 | 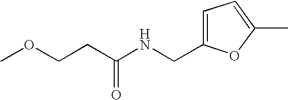 | 0.8 |
| | N-[(5-methyl-2-furyl)methyl]-3- | |
| | (methylsulfanyl)propanamide | |
| 2 | 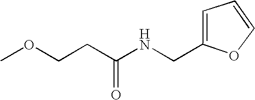 | 9.8 |
| | N-(2-furylmethyl)-3-(methylsulfanyl)propanamide | |
| 3 | 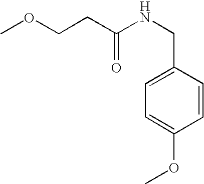 | 6 |
| | N-(4-methoxybenzyl)-3- | |
| | (methylsulfanyl)propanamide | |
| 4 | 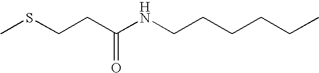 | 12 |
| | N-hexyl-3-(methylsulfanyl)propanamide | |
| 5 | 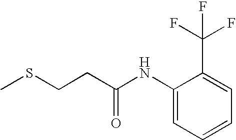 | |
| | 3-(methylsulfanyl)-N-[2- | |
| | (trifluoromethyl)phenyl]propanamide | |
| 6 | 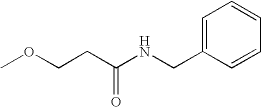 | 1.6 |
| | N-benzyl-3-(methylsulfanyl)propanamide | |
| 7 | 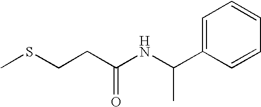 | 14.7 |
| | 3-(methylsulfanyl)-N-(1-phenylethyl)propanamide | |
| 8 | 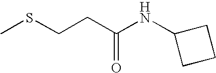 | 47 |
| | N-cyclobutyl-3-(methylsulfanyl)propanamide | |

Furthermore, compound 5 inhibits 15-PGDH to a proportion of 21% at 100 μM.

From this table, it is seen that compounds 1 to 8 are indeed 15-PGDH inhibitors. Furthermore, they inhibit 15-PGDH more efficiently and more selectively than PGFS: the IC_{50dh} is lower than the IC_{50fs}.

The compositions below are obtained via the usual techniques commonly used in the cosmetic or pharmaceutical field.

In each of the formulation examples, a fluid is obtained that may be used as an application once or twice a day to the scalp, to reduce hair loss and/or to promote hair growth.

### Example 18

### Hair Lotion

| | | |
|---|---|---|
| Compound of Example 1 | 1.00 | g |
| Propylene glycol | 30.00 | g |
| Ethyl alcohol | 40.00 | g |
| Water | qs 100.00 | g |

This lotion is applied to the scalp, once or twice a day, at a rate of 1 mL per application. This lotion makes it possible to reduce hair loss and to promote hair regrowth; it also makes it possible to repigment the hair and thus to hide the white and/or grey hairs.

### Example 19

### Hair Lotion

| | | |
|---|---|---|
| Compound 1 | 1.00 | g |
| Propylene glycol | 30.00 | g |
| Ethyl alcohol | 40.00 | g |
| Water | qs 100.00 | g |

This lotion is applied to the scalp, once or twice a day, at a rate of 1 mL per application, by gently massaging the scalp to make the active agent penetrate in. The head of hair is then dried in the open air. This lotion makes it possible to reduce hair loss and to promote regrowth of the hair, and also to repigment grey and/or white hairs.

### Example 20

### Wax/Water Mascara

| | |
|---|---|
| Beeswax | 6.00% |
| Paraffin wax | 13.00% |
| Hydrogenated jojoba oil | 2% |
| Water-soluble film-forming polymer | 3% |
| Triethanolamine stearate | 8% |
| Compound 1 | 1% |
| Black pigment | 5% |
| Preservative | qs |
| Water | qs 100% |

This mascara is applied to the eyelashes like a standard mascara with a mascara brush. It promotes the regrowth and the repigmentation of the eyelashes.

### Example 21

### Hair Lotion

| | | |
|---|---|---|
| Compound of Example 1 | 1.00 | g |
| Latanoprost | 0.10 | g |
| Propylene glycol | 30.00 | g |
| Ethyl alcohol | 40.00 | g |
| Water | qs 100.00 | g |

This lotion is applied to the scalp, once or twice a day, at a rate of 1 mL per application, by gently massaging the scalp to make the active agents penetrate in. The head of hair is then dried in the open air. This lotion makes it possible to reduce hair loss and to promote regrowth of the hair while at the same time repigmenting it.

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for inducing and/or stimulating the growth of keratin fibers and/or for stopping the loss and/or increasing the density thereof, comprising administering to a subject in need of such treatment, a thus effective amount of at least one 3-sulfanylpropanamide compound of formula (I) or a salt and/or solvate thereof: 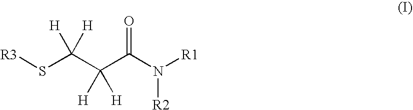
in which:
a) R₁ and R₂ independently represent:
1) a hydrogen atom,
2) a C₁-C₂₀ alkyl radical optionally substituted with at least one substituent A₁,
3) a saturated hydrocarbon-based ring C¹ optionally substituted with at least one substituent A₁;
4) an unsaturated hydrocarbon-based ring C² optionally substituted with at least one substituent A₂;
b) R₃ is:
1) a hydrogen atom,
2) a C₁-C₂₀ alkyl radical optionally substituted with at least one substituent A₃,
3) a saturated hydrocarbon-based ring C³ optionally substituted with at least one substituent A₄;
c) A₁ is:
1) a fluorine atom,
2) a group CF₃, CN, OR, SR, NRR′, SO₂NRR′, NRSO₂R′ or SO₂R,
3) a group having the following formula: 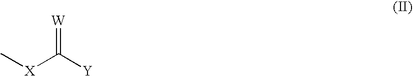
wherein:
W=O, NH, S,
X=O, NH, C₁-C₆ N-alkyl,
Y=C₁-C₄ alkyl, C₁-C₆ alkoxy, NH₂, C₁-C₆ NH-alkyl, C₁-C₆ N-dialkyl,
4) a saturated hydrocarbon-based ring C⁴ optionally substituted with at least one substituent A₄,
5) a heterocycle Hy₁ selected from the group consisting of pyrrole, furan, dihydrofuran, tetrahydrofuran, thiophene, thiazole, oxazole, oxazolidine, isoxazole, isoxazolidine, oxadiazole, pyridine, pyran, dihydropyran, tetrahydropyran, pyrimidine, piperazine, pyridazine, pyrazine, morpholine, quinoline, isoquinoline, 2-thiohydantoin, 2-oxo-2,3-dihydro-1,3thiazole and pyridone, such heterocycle optionally being substituted with at -least one substituent A₄,
6) an unsaturated hydrocarbon-based ring C³ optionally substituted with at least one substituent A₄ and optionally fused to a hydrocarbon-based or heterocyclic ring C⁶;
d) A₂ is:
1) a fluorine atom,
2) a group of formula CF₃, CN, OR, SR, NRR′, COR, COOR, CONRR′, NRCOR′, NRCONR′R″, SO₂NRR′, NRSO₂R′ or SO₂R,
3) a C₁-C₂₀ alkyl radical optionally substituted with at least one substituent A₄;
e) A₃ is:
1) a fluorine atom,
2) a group CF₃, CN, OR, SR, NRR′,
3) a group of formula (II) 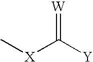
wherein W, X and Y have the same definitions as above,
4) a saturated hydrocarbon-based ring C⁴ optionally substituted with at least one substituent A₄,
5) an unsaturated hydrocarbon-based ring C⁵ optionally substituted with at least one substituent A₄ or a heterocycle Hy₂ optionally substituted with at least one substituent A₄ and containing at least one hetero atom selected from among N, O and S and combinations thereof;
f) A₄ is:
1) a fluorine atom,
2) a group CF₃, CN, OR, SR, NRR′,
3) a C₁-C₂₀ alkyl radical, or
4) a ring C⁶ optionally fused to another ring C⁷, these rings optionally containing at least one hetero atom to form a heterocycle Hy₂;
g) R, R′ and R″, which may be identical or different, are each:
1) a hydrogen atom,
2) a saturated C₁-C₆ alkyl radical optionally substituted with at least one substituent A₄, or
3) an unsaturated hydrocarbon-based ring C⁵ optionally substituted with at least one substituent A₄,
4) a heterocycle Hy₂ comprising at least one hetero atom selected from among N, S and O or a combination thereof and optionally being substituted with at least one substituent A₄.

2. A cosmetic care and/or makeup composition for keratin fibers to induce and/or stimulate the growth and/or to stop the loss and/or increase the density thereof, comprising a thus effective amount of at least one 3-sulfanylpropanamide compound of formula (I) or a salt and/or solvate thereof: 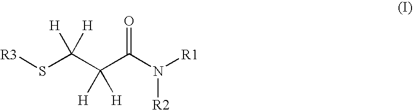
in which:
a) R₁ and R₂ independently represent:
1) a hydrogen atom,
2) a C₁-C₂₀ alkyl radical optionally substituted with at least one substituent A₁,
3) a saturated hydrocarbon-based ring C¹ optionally substituted with at least one substituent A₁;
4) an unsaturated hydrocarbon-based ring C² optionally substituted with at least one substituent A₂;
b) R₃ is:
1) a hydrogen atom,
2) a C₁-C₂₀ alkyl radical optionally substituted with at least one substituent A₃,
3) a saturated hydrocarbon-based ring C³ optionally substituted with at least one substituent A₄;
c) A₁ is:
1) a fluorine atom,
2) a group CF₃, CN, OR, SR, NRR′, SO₂NRR′, NRSO₂R′ or SO₂R,
3) a group having the following formula: 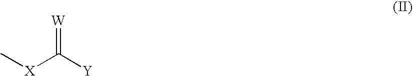
wherein:
W=O, NH, S,
X=O, NH, C₁-C₆ N-alkyl,
Y=C₁-C₄ alkyl, C₁-C₆ alkoxy, NH₂, C₁-C₆ NH-alkyl, C₁-C₆ N-dialkyl,
4) a saturated hydrocarbon-based ring C⁴ optionally substituted with at least one substituent A₄,
5) a heterocycle Hy, selected from the group consisting of pyrrole, furan, dihydrofuran, tetrahydrofuran, thiophene, thiazole, oxazole, oxazolidine, isoxazole, isoxazolidine, oxadiazole, pyridine, pyran, dihydropyran, tetrahydropyran, pyrimidine, piperazine, pyridazine, pyrazine, morpholine, quinoline, isoquinoline, 2-thiohydantoin, 2-oxo-2,3-dihydro-1,3-thiazole and pyridone, such heterocycle optionally being substituted with at least one substituent A₄,
6) an unsaturated hydrocarbon-based ring C³ optionally substituted with at least one substituent A₄ and optionally fused to a hydrocarbon-based or heterocyclic ring C⁶;
d) A₂ is:
1) a fluorine atom,
2) a group of formula CF₃, CN, OR, SR, NRR′, COR, COOR, CONRR′, NRCOR′, NRCONR′R″, SO₂NRR′, NRSO₂R′ or SO₂R,
3) a C₁-C₂₀ alkyl radical optionally substituted with at least one substituent A₄;
e) A₃ is:
1) a fluorine atom,
2) a group CF₃, CN, OR, SR, NRR′,
3) a group of formula (II) 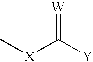
wherein W, X and Y have the same definitions as above,
4) a saturated hydrocarbon-based ring C⁴ optionally substituted with at least one substituent A₄,
5) an unsaturated hydrocarbon-based ring C⁵ optionally substituted with at least one substituent A₄ or a heterocycle Hy₂ optionally substituted with at least one substituent A₄ and containing at least one hetero atom selected from among N, O and S and combinations thereof;
A₄ is:
1) a fluorine atom,
2) a group CF₃, CN, OR, SR, NRR′,
3) a C₁-C₂₀ alkyl radical, or
4) a ring C⁶ optionally fused to another ring C⁷, these rings optionally containing at least one hetero atom to form a heterocycle Hy₂;
g) R, R′ and R″, which may be identical or different, are each:
1) a hydrogen atom,
2) a saturated C₁-C₆ alkyl radical optionally substituted with at least one substituent A₄, or
3) an unsaturated hydrocarbon-based ring C⁵ optionally substituted with at least one substituent A₄,
4) a heterocycle Hy₂ comprising at least one hetero atom selected from among N, S and O or a combination thereof and optionally being substituted with at least one substituent A₄, formulated into a cosmetically/physiologically acceptable medium therefor.

3. A regime or regimen for inducing and/or stimulating the pigmentation of the skin and/or keratin fibers and/or for limiting the depigmentation and/or bleaching thereof, comprising administering to a subject in need of such treatment, a thus effective amount of at least one 3-sulfanylpropanamide compound of formula (I) or a salt and/or solvate thereof: 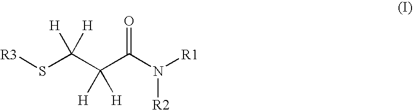
in which:
a) R₁ and R₂ independently represent:
1) a hydrogen atom,
2) a C₁-C₂₀ alkyl radical optionally substituted with at least one substituent A₁,
3) a saturated hydrocarbon-based ring C¹ optionally substituted with at least one substituent A₁;
4) an unsaturated hydrocarbon-based ring C² optionally substituted with at least one substituent A₂;
b) R₃ is:
1) a hydrogen atom,
2) a C₁-C₂₀ alkyl radical optionally substituted with at least one substituent A₃,
3) a saturated hydrocarbon-based ring C³ optionally substituted with at least one substituent A₄;
c) A₁ is:
1) a fluorine atom,
2) a group CF₃, CN, OR, SR, NRR′, SO₂NRR′, NRSO₂R′ or SO₂R,
3) a group having the following formula: 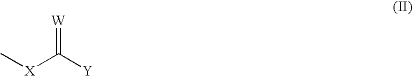
wherein:
W=O, NH, S,
X=O, NH, C₁-C₆ N-alkyl,
Y=C₁-C₄ alkyl, C₁-C₆ alkoxy, NH₂, C₁-C₆ NH-alkyl, C₁-C₆ N-dialkyl,
4) a saturated hydrocarbon-based ring C⁴ optionally substituted with at least one substituent A₄,
5) a heterocycle Hy₁ selected from the group consisting of pyrrole, furan, dihydrofuran, tetrahydrofuran, thiophene, thiazole, oxazole, oxazolidine, isoxazole, isoxazolidine, oxadiazole, pyridine, pyran, dihydropyran, tetrahydropyran, pyrimidine, piperazine, pyridazine, pyrazine, morpholine, quinoline, isoquinoline, 2-thiohydantoin, 2-oxo-2,3-dihydro-1,3-thiazole and pyridone, such heterocycle optionally being substituted with at least one substituent A₄,
6) an unsaturated hydrocarbon-based ring C³ optionally substituted with at least one substituent A₄ and optionally fused to a hydrocarbon-based or heterocyclic ring C⁶;
d) A₂ is:
1) a fluorine atom,
2) a group of formula CF₃, CN, OR, SR, NRR′, COR, COOR, CONRR′, NRCOR′, NRCONR′R″, SO₂NRR′, NRSO₂R′ or SO₂R,
3) a C₁-C₂₀ alkyl radical optionally substituted with at least one substituent A₄;
e) A₃ is:
1) a fluorine atom,
2) a group CF₃, CN, OR, SR, NRR′,
3) a group of formula (II) 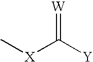
wherein W, X and Y have the same definitions as above,
4) a saturated hydrocarbon-based ring C⁴ optionally substituted with at least one substituent A₄,
5) an unsaturated hydrocarbon-based ring C⁵ optionally substituted with at least one substituent A₄ or a heterocycle Hy₂ optionally substituted with at least one substituent A₄ and containing at least one hetero atom selected from among N, O and S and combinations thereof;
f) A₄ is:
1) a fluorine atom,
2) a group CF₃, CN, OR, SR, NRR′,
3) a C₁-C₂₀ alkyl radical, or
4) a ring C⁶ optionally fused to another ring C⁷, these rings optionally containing at least one hetero atom to form a heterocycle Hy₂;
g) R, R′ and R″, which may be identical or different, are each:
1) a hydrogen atom,
2) a saturated C₁-C₆ alkyl radical optionally substituted with at least one substituent A₄, or
3) an unsaturated hydrocarbon-based ring C⁵ optionally substituted with at least one substituent A₄,
4) a heterocycle Hy₂ comprising at least one hetero atom selected from among N, S and O or a combination thereof and optionally being substituted with at least one substituent A₄.

4. A cosmetic/pharmaceutical composition useful to induce and/or stimulate the pigmentation of the skin and/or keratin fibers and/or to limit the depigmentation and/or bleaching thereof, comprising a thus effective amount of at least one 3-sulfanylpropanamide compound of formula (I) or a salt and/or solvate thereof: 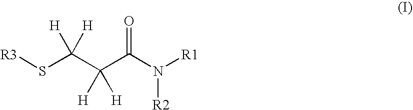
in which:
a) R₁ and R₂ independently represent:
1) a hydrogen atom,
2) a C₁-C₂₀ alkyl radical optionally substituted with at least one substituent A₁,
3) a saturated hydrocarbon-based ring C¹ optionally substituted with at least one substituent A₁;
4) an unsaturated hydrocarbon-based ring C² optionally substituted with at least one substituent A₂;
b) R₃ is:
1) a hydrogen atom,
2) a C₁-C₂₀ alkyl radical optionally substituted with at least one substituent A₃,
3) a saturated hydrocarbon-based ring C³ optionally substituted with at least one substituent A₄;
c) A₁ is:
1) a fluorine atom,
2) a group CF₃, CN, OR, SR, NRR′, SO₂NRR′, NRSO₂R′ or SO₂R,
3) a group having the following formula: 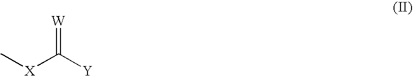
wherein:
W=O, NH, S,
X=O, NH, C₁-C₆ N-alkyl,
Y=₁-C₄ alkyl, C₁-C₆ alkoxy, NH₂, C₁-C₆ NH-alkyl, C₁-C₆ N-dialkyl,
4) a saturated hydrocarbon-based ring C⁴ optionally substituted with at least one substituent A₄,
5) a heterocycle Hy₁ selected from the group consisting of pyrrole, furan, dihydrofuran, tetrahydrofuran, thiophene, thiazole, oxazole, oxazolidine, isoxazole, isoxazolidine, oxadiazole, pyridine, pyran, dihydropyran, tetrahydropyran, pyrimidine, piperazine, pyridazine, pyrazine, morpholine, quinoline, isoquinoline, 2-thiohydantoin, 2-oxo-2,3-dihydro-1,3-thiazole and pyridone, such heterocycle optionally being substituted with at least one substituent A₄,
6) an unsaturated hydrocarbon-based ring C³ optionally substituted with at least one substituent A₄ and optionally fused to a hydrocarbon-based or heterocyclic ring C⁶;
d) A₂ is:
1) a fluorine atom,
2) a group of formula CF₃, CN, OR, SR, NRR′, COR, COOR, CONRR′, NRCOR′, NRCONR′R″, SO₂NRR′, NRSO₂R′ or SO₂R,
3) a C₁-C₂₀ alkyl radical optionally substituted with at least one substituent A₄;
e) A₃ is:
1) a fluorine atom,
2) a group CF₃, CN, OR, SR, NRR′,
3) a group of formula (II) 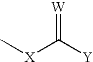
wherein W, X and Y have the same definitions as above,
4) a saturated hydrocarbon-based ring C⁴ optionally substituted with at least one substituent A₄,
5) an unsaturated hydrocarbon-based ring C⁵ optionally substituted with at least one substituent A₄ or a heterocycle Hy₂ optionally substituted with at least one substituent A₄ and containing at least one hetero atom selected from among N, O and S and combinations thereof;
f) A₄ is:
1) a fluorine atom,
2) a group CF₃, CN, OR, SR, NRR′,
3) a C₁-C₂₀ alkyl radical, or
4) a ring C⁶ optionally fused to another ring C⁷, these rings optionally containing at least one hetero atom to form a heterocycle Hy₂;
g) R, R′ and R″, which may be identical or different, are each:
1) a hydrogen atom,
2) a saturated C₁-C₆ alkyl radical optionally substituted with at least one substituent A₄, or
3) an unsaturated hydrocarbon-based ring C⁵ optionally substituted with at least one substituent A₄,
4) a heterocycle Hy₂ comprising at least one hetero atom selected from among N, S and O or a combination thereof and optionally being substituted with at least one substituent A₄, formulated into a cosmetically/pharmaceutically acceptable medium therefor.

5. A regime or regimen for inhibiting 15-hydroxyprostaglandin dehydrogenase, comprising administering to a subject in need of such treatment, a thus effective amount of at least one 3-sulfanylpropanamide compound of formula (I) or a salt and/or solvate thereof: 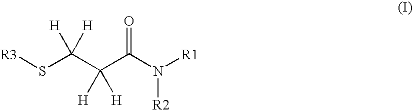
in which:
a) R₁ and R₂ independently represent:
1) a hydrogen atom,
2) a C₁-C₂₀ alkyl radical optionally substituted with at least one substituent A₁,
3) a saturated hydrocarbon-based ring C¹ optionally substituted with at least one substituent A₁;
4) an unsaturated hydrocarbon-based ring C² optionally substituted with at least one substituent A₂;
b) R₃ is:
1) a hydrogen atom,
2) a C₁-C₂₀ alkyl radical optionally substituted with at least one substituent A₃,
3) a saturated hydrocarbon-based ring C³ optionally substituted with at least one substituent A₄;
c) A₁ is:
1) a fluorine atom,
2) a group CF₃, CN, OR, SR, NRR′, SO₂NRR′, NRSO₂R′ or SO₂R,
3) a group having the following formula: 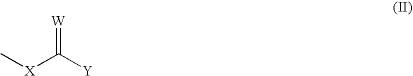
wherein:
W=O, NH, S,
X=O, NH, C₁-C₆ N-alkyl,
Y=C₁-C₄ alkyl, C₁-C₆ alkoxy, NH₂, C₁-C₆ NH-alkyl, C₁-C₆ N-dialkyl,
4) a saturated hydrocarbon-based ring C⁴ optionally substituted with at least one substituent A₄,
5) a heterocycle Hy₁selected from the group consisting of pyrrole, furan, dihydrofuran, tetrahydrofuran, thiophene, thiazole, oxazole, oxazolidine, isoxazole, isoxazolidine, oxadiazole, pyridine, pyran, dihydropyran, tetrahydropyran, pyrimidine, piperazine, pyridazine, pyrazine, morpholine, quinoline, isoquinoline, 2-thiohydantoin, 2-oxo-2,3-dihydro-1,3-thiazole and pyridone, such heterocycle optionally being substituted with at least one substituent A₄,
6) an unsaturated hydrocarbon-based ring C³ optionally substituted with at least one substituent A₄ and optionally fused to a hydrocarbon-based or heterocyclic ring C⁶;
d) A₂ is:
1) a fluorine atom,
2) a group of formula CF₃, CN, OR, SR, NRR′, COR, COOR, CONRR′, NRCOR′, NRCONR′R″, SO₂NRR′, NRSO₂R′ or SO₂R,
3) a C₁-C₂₀ alkyl radical optionally substituted with at least one substituent A₄;
e) A₃ is:
1) a fluorine atom,
2) a group CF₃, CN, OR, SR, NRR′,
3) a group of formula (II) 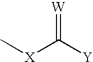
wherein W, X and Y have the same definitions as above,
4) a saturated hydrocarbon-based ring C⁴ optionally substituted with at least one substituent A₄,
5) an unsaturated hydrocarbon-based ring C⁵ optionally substituted with at least one substituent A₄ or a heterocycle Hy₂ optionally substituted with at least one substituent A4 and containing at least one hetero atom selected from among N, O and S and combinations thereof;
f) A₄ is:
1) a fluorine atom,
2) a group CF₃, CN, OR, SR, NRR′,
3) a C₁-C₂₀ alkyl radical, or
4) a ring C⁶ optionally fused to another ring C⁷, these rings optionally containing at least one hetero atom to form a heterocycle Hy₂;
g) R, R′ and R″, which may be identical or different, are each:
1) a hydrogen atom,
2) a saturated C₁-C₆ alkyl radical optionally substituted with at least one substituent A₄, or
3) an unsaturated hydrocarbon-based ring C⁵ optionally substituted with at least one substituent A₄,
4) a heterocycle Hy₂ comprising at least one hetero atom selected from among N, S and O or a combination thereof and optionally being substituted with at least one substituent A₄.

6. The regime or regimen as defined by claim 5, for inhibiting type 1 15-hydroxyprostaglandin dehydrogenase.

7. The regime or regimen as defined by claim 1, for inducing and/or stimulating and/or for stopping the loss and/or increasing the density of human head hair, the eyebrows, the eyelashes, beard hair, moustache hair and pubic hair, and the nails.

8. A regime or regimen to reduce human hair loss and/or to increase the density thereof and/or to treat andro-chrono-genetic alopecia and/or to treat alopecia of natural origin and/or to reduce the depigmentation and/or bleaching thereof, comprising administering to an individual in need of such treatment, a thus effective amount of at least one 3-sulfanylpropanamide compound of formula (I) or a salt and/or solvate thereof: 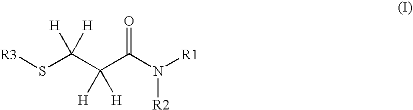
in which:
a) R₁ and R₂ independently represent:
1) a hydrogen atom,
2) a C₁-C₂₀ alkyl radical optionally substituted with at least one substituent A₁,
3) a saturated hydrocarbon-based ring C¹ optionally substituted with at least one substituent A₁;
4) an unsaturated hydrocarbon-based ring C² optionally substituted with at least one substituent A₂;
b) R₃ is:
1) a hydrogen atom,
2) a C₁-C₂₀ alkyl radical optionally substituted with at least one substituent A₃,
3) a saturated hydrocarbon-based ring C³ optionally substituted with at least one substituent A₄;
c) A₁ is:
1) a fluorine atom,
2) a group CF₃, CN, OR, SR, NRR′, SO₂NRR′, NRSO₂R′ or SO₂R,
3) a group having the following formula: 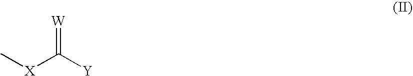
wherein:
W=O, NH, S,
X=O, NH, C₁-C₆ N-alkyl,
Y=C₁-C₄ alkyl, C₁-C₆ alkoxy, NH₂, C₁-C₆ NH-alkyl, C₁-C₆ N-dialkyl,
4) a saturated hydrocarbon-based ring C⁴ optionally substituted with at least one substituent A₄,
5) a heterocycle Hy₁ selected from the group consisting of pyrrole, furan, dihydrofuran, tetrahydrofuran, thiophene, thiazole, oxazole, oxazolidine, isoxazole, isoxazolidine, oxadiazole, pyridine, pyran, dihydropyran, tetrahydropyran, pyrimidine, piperazine, pyridazine, pyrazine, morpholine, quinoline, isoquinoline, 2-thiohydantoin, 2-oxo-2,3-dihydro-1,3-thiazole and pyridone, such heterocycle optionally being substituted with at least one substituent A₄,
6) an unsaturated hydrocarbon-based ring C³ optionally substituted with at least one substituent A₄ and optionally fused to a hydrocarbon-based or heterocyclic ring C⁶;
d) A₂ is:
1) a fluorine atom,
2) a group of formula CF₃, CN, OR, SR, NRR′, COR, COOR, CONRR′, NRCOR′, NRCONR′R″, SO₂NRR′, NRSO₂R′ or SO₂R,
3) a C₁-C₂₀ alkyl radical optionally substituted with at least one substituent A₄;
e) A₃ is:
1) a fluorine atom,
2) a group CF₃, CN, OR, SR, NRR′,
3) a group of formula (II) 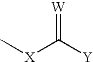
wherein W, X and Y have the same definitions as above,
4) a saturated hydrocarbon-based ring C⁴ optionally substituted with at least one substituent A₄,
5) an unsaturated hydrocarbon-based ring C⁵ optionally substituted with at least one substituent A₄ or a heterocycle Hy₂ optionally substituted with at least one substituent A₄ and containing at least one hetero atom selected from among N, O and S and combinations thereof;
f) A₄ is:
1) a fluorine atom,
2) a group CF₃, CN, OR, SR, NRR′,
3) a C₁-C₂₀ alkyl radical, or
4) a ring C⁶ optionally fused to another ring C⁷, these rings optionally containing at least one hetero atom to form a heterocycle Hy₂;
g) R, R′ and R″, which may be identical or different, are each:
1) a hydrogen atom,
2) a saturated C₁-C₆ alkyl radical optionally substituted with at least one substituent A₄, or
3) an unsaturated hydrocarbon-based ring C⁵ optionally substituted with at least one substituent A₄,
4) a heterocycle Hy₂ comprising at least one hetero atom selected from among N, S and O or a combination thereof and optionally being substituted with at least one substituent A₄.

9. A regime or regimen to induce and/or stimulate the growth of human eyelashes and/or to increase the density and/or to induce and/or stimulate the pigmentation and/or to limit the depigmentation and/or reduce the bleaching thereof, comprising administering to an individual in need of such treatment, a thus effective amount of at least one 3-sulfanylpropanamide compound of formula (I) or a salt and/or solvate thereof: 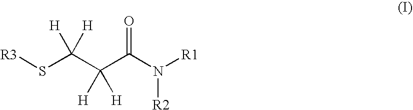
in which:
a) R₁ and R₂ independently represent:
1) a hydrogen atom,
2) a C₁-C₂₀ alkyl radical optionally substituted with at least one substituent A₁,
3) a saturated hydrocarbon-based ring C¹ optionally substituted with at least one substituent A₁;
4) an unsaturated hydrocarbon-based ring C² optionally substituted with at least one substituent A₂;
b) R₃ is:
1) a hydrogen atom,
2) a C₁-C₂₀ alkyl radical optionally substituted with at least one substituent A₃,
3) a saturated hydrocarbon-based ring C³ optionally substituted with at least one substituent A₄;
c) A₁ is:
1) a fluorine atom,
2) a group CF₃, CN, OR, SR, NRR′, SO₂NRR′, NRSO₂R′ or SO₂R,
3) a group having the following formula: 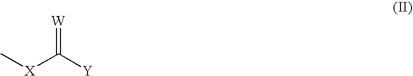
wherein:
W=O, NH, S,
X=O, NH, C₁-C₆ N-alkyl,
Y=C₁-C₄ alkyl, C₁-C₆ alkoxy, NH₂, C₁-C₆ NH-alkyl, C₁-C₆ N-dialkyl,
4) a saturated hydrocarbon-based ring C⁴ optionally substituted with at least one substituent A₄,
5) a heterocycle Hy₁ selected from the group consisting of pyrrole, furan, dihydrofuran, tetrahydrofuran, thiophene, thiazole, oxazole, oxazolidine, isoxazole, isoxazolidine, oxadiazole, pyridine, pyran, dihydropyran, tetrahydropyran, pyrimidine, piperazine, pyridazine, pyrazine, morpholine, quinoline, isoquinoline, 2-thiohydantoin, 2-oxo-2,3-dihydro-1,3-thiazole and pyridone, such heterocycle optionally being substituted with at least one substituent A₄,
6) an unsaturated hydrocarbon-based ring C³ optionally substituted with at least one substituent A₄ and optionally fused to a hydrocarbon-based or heterocyclic ring C⁶;
d) A₂ is:
1) a fluorine atom,
2) a group of formula CF₃, CN, OR, SR, NRR′, COR, COOR, CONRR′, NRCOR′, NRCONR′R″, SO₂NRR′, NRSO₂R′ or SO₂R,
3) a C₁-C₂₀ alkyl radical optionally substituted with at least one substituent A₄;
e) A₃ is:
1) a fluorine atom,
2) a group CF₃, CN, OR, SR, NRR′,
3) a group of formula (II) 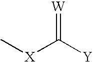
wherein W, X and Y have the same definitions as above,
4) a saturated hydrocarbon-based ring C⁴ optionally substituted with at least one substituent A₄,
5) an unsaturated hydrocarbon-based ring C⁵ optionally substituted with at least one substituent A₄ or a heterocycle Hy₂ optionally substituted with at least one substituent A₄ and containing at least one hetero atom selected from among N, O and S and combinations thereof;
A₄ is:
1) a fluorine atom,
2) a group CF₃, CN, OR, SR, NRR′,
3) a C₁-C₂₀ alkyl radical, or
4) a ring C⁶ optionally fused to another ring C⁷, these rings optionally containing at least one hetero atom to form a heterocycle Hy₂;
g) R, R′ and R″, which may be identical or different, are each:
1) a hydrogen atom,
2) a saturated C₁-C₆ alkyl radical optionally substituted with at least one substituent A₄, or
3) an unsaturated hydrocarbon-based ring C⁵ optionally substituted with at least one substituent A₄,
4) a heterocycle Hy₂ comprising at least one hetero atom selected from among N, S and O or a combination thereof and optionally being substituted with at least one substituent A₄.

10. The regime or regimen as defined by claim 1, said at least one 3-sulfanylpropanamide compound of formula (I) or salt and/or solvate thereof comprising at least one heterocycle Hy₂ selected from the group consisting of azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, dihydrothiazole, thiazole, dihydrothiazole, thiazolidine, pyrazole, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine, and diazepine.

11. The regime or regimen as defined by claim 10, said at least one heterocycle Hy₂ being selected from the group consisting of pyrrole, furan, dihydrofuran, tetrahydrofuran, thiophene, thiazole, oxazole, oxazolidine, isoxazole, isoxazolidine, oxadiazole, pyridine, pyran, dihydropyran, tetrahydropyran, pyrimidine, piperazine, pyridazine, pyrazine, morpholine, quinoline, isoquinoline, 2-thiohydantoin, 2-oxo-2,3-dihydro-1,3-thiazole and pyridone.

12. The regime or regimen as defined by claim 1, wherein said at least one 3-sulfanylpropanamide compound of formula (I) or salt and/or solvate thereof, one of R₁ and R₂ is hydrogen.

13. The regime or regimen as defined by claim 1, wherein formula (I) or salt and/or solvate thereof, R₁ is a linear or branched, saturated or unsaturated C₁ to C₁₀ alkyl radical or a saturated or unsaturated hydrocarbon-based ring, such radical or such ring being substituted with an unsaturated hydrocarbon-based or heterocyclic ring C⁵, such ring C⁵ itself being optionally substituted with one or more substituents A₁.

14. The regime or regimen as defined by claim 1, said at least one 3-sulfanylpropanamide compound of formula (I) or salt and/or solvate thereof comprising at least one substituent A, selected from the group consisting of:
linear or branched, saturated or unsaturated C₁-C₁₀ alkyl radicals;
C₁-C₆ alkoxy radicals;
aryl rings.

15. The regime or regimen as defined by claim 14, said at least one substituent A₁ being selected from the group consisting of methyl, ethyl and methoxy radicals and phenyl and naphthyl rings.

16. The regime or regimen as defined by claim 1, wherein formula (I) or salt and/or solvate thereof, R₃ is a linear or branched, saturated or unsaturated C₁ to C₁₀ alkyl radical or a saturated or unsaturated hydrocarbon-based ring.

17. The regime or regimen as defined by claim 16, wherein R₃ is a methyl, tert-butyl, cyclopentyl or cyclohexyl radical.

18. The regime or regimen as defined by claim 1, comprising a hydrate of said at least one 3-sulfanylpropanamide compound of formula (I).

19. The regime or regimen as defined by claim 1, said at least one 3-sulfanylpropanamide compound having the formula (Ia) or salt and/or solvate thereof:

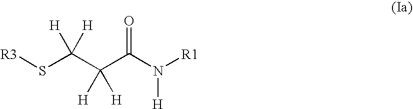

20. The regime or regimen as defined by claim 1, said at least one 3-sulfanylpropanamide compound having one of the formulae (III), (IV), (V), (VII) and (VIII) below: 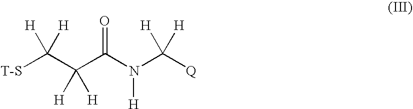
in which Q is:
a) a phenyl radical substituted with at least one radical Z; or
b) an aromatic heterocycle selected from the group consisting of pyrimidine, quinoline, isoquinoline, pyrrole, furan and thiophene, optionally substituted with at least one radical Z,
wherein Z is:
fluorine,
a group CF₃, CN, OR, SR, NRR′, SO₂NRR′, NRSO₂R′, SO₂R SiRR′R″,
a group of formula (II): 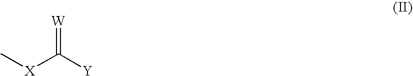
a C₁-C₁₀ alkyl radical optionally substituted with at least one substituent A₄;
and in which T is:
a) a saturated alkyl radical selected among from methyl, ethyl, n-butyl, isobutyl and tert-butyl; or
b) a saturated C₄-C₆ hydrocarbon-based ring:

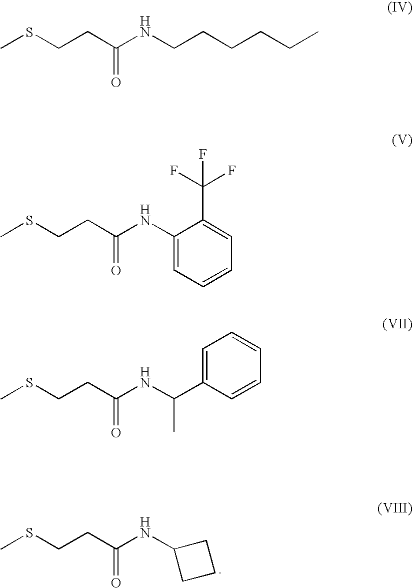

21. The regime or regimen as defined by claim 1, comprising a salt of a 3-sulfanylpropanamide compound of formula (I) selected from the group consisting of the sodium and potassium salts, the ammonium salts, the zinc (Zn²⁺), calcium (Ca²⁺), copper (Cu²⁺), iron (Fe²⁺and Fe³⁺), strontium (Sr²⁺), magnesium (Mg²⁺) and manganese (Mn²⁺) salts, and the triethanolamine, monoethanolamine, diethanolamine, hexadecylamine, N,N,N′,N′-tetrakis(2-hydroxypropyl)ethylenediamine, tris-hydroxymethylaminomethane, hydroxide, hydrohalide, carbonate, hydrogen carbonate, citrate, lactate, glycolate, gluconate, acetate, propionate, fumarate, oxalate, tartrate, sulfate, phosphate and hydrogen phosphate salts.

22. The regime or regimen as defined by claim 1, said at least one 3-sulfanylpropanamide compound of formula (I) having one of the following formulae:
Compound 1: N-[(5-methyl-2-furyl)methyl]-3-(methylsulfanyl)propanamide: 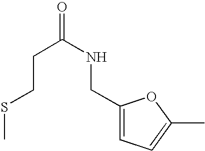
Compound 2: N-(2-furylmethyl)-3-(methylsulfanyl)propanamide: 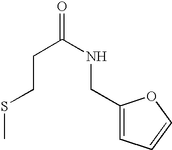
Compound 3: N-(4-methoxybenzyl)-3-(methylsulfanyl)propanamide: 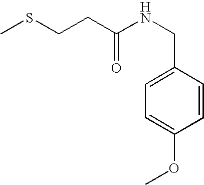
Compound 4: N-hexyl-3-(methylsulfanyl)propanamide: 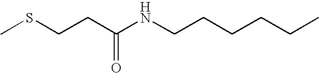
Compound 5: 3-(methylsulfanyl)-N-[2-(trifluoromethyl)phenyl]propanamide: 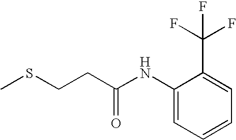
Compound 6: N-benzyl-3-(methylsulfanyl)propanamide: 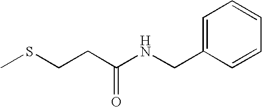
Compound 7: 3-(methylsulfanyl)-N-(1-phenylethyl)propanamide: 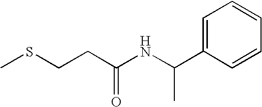
Compound 8: N-cyclobutyl-3-(methylsulfanyl)propanamide:

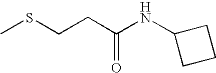

23. A topically applicable composition for caring for and/or making up the skin and/or keratin fibers, comprising a thus effective amount of at least one 3-sulfanylpropanamide compound having one of the formulae (III), (IV), (V), (VII), and (VIII) or a salt and/or solvate thereof: 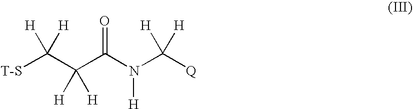
wherein Q is:
a) a phenyl radical substituted with at least one radical Z; or
b) an aromatic heterocycle selected from the group consisting of pyrimidine, quinoline, isoquinoline, pyrrole, furan or thiophene, optionally substituted with at least one radical Z,
wherein Z is:
fluorine,
a group CF₃, CN, OR, SR, NRR′, SO₂NRR′, NRSO₂R′, SO₂R, SiRR′R″,
a group of formula (II): 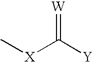
wherein:
W=O, NH, S,
X=O, NH, C₁-C₆ N-alkyl,
Y=C₁-C₄ alkyl, C₁-C₆ alkoxy, NH₂, C₁-C₆ NH-alkyl, C₁-C₆ N-dialkyl,
a C₁-C₁₀ alkyl radical optionally substituted with at least one substituent A₄;
R, R′ and R″, which may be identical or different, are each:
1) a hydrogen atom,
2) a saturated C₁-C₆ alkyl radical optionally substituted with at least one substituent A₄, or
3) an unsaturated hydrocarbon-based ring C₅ optionally substituted with at least one substituent A₄,
4) a heterocycle Hy₂ comprising at least one hetero atom selected from among N, S and O or a combination thereof and optionally being substituted with at least one substituent A₄,
wherein A₄ is:
1) a fluorine atom,
2) a group CF₃, CN, OR, SR, NRR′
3) a C₁-C₂₀ alkyl radical, or
4) a ring C⁶ optionally fused to another ring C⁷, such rings optionally containing at least one hetero atom to form a heterocycle Hy₂, wherein T is:
a) a saturated alkyl radical selected from among methyl, ethyl, n-butyl, isobutyl and tert-butyl; or
b) a saturated C₄-C₆ hydrocarbon-based ring; 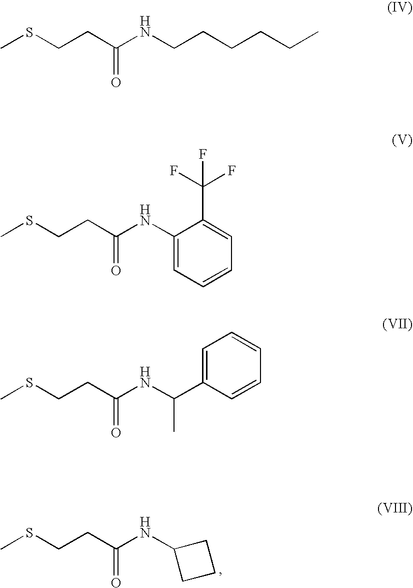
formulated into a topically applicable, physiologically acceptable medium therefor.

24. The topically applicable composition as defined by claim 23, said at least one 3-sulfanylpropanamide compound have at least one of the following formulae:
Compound 1: N-[(5-methyl-2-furyl)methyl]-3-(methylsulfanyl)propanamide: 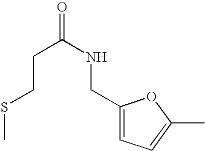
Compound 2: N-(2-furylmethyl)-3-(methylsulfanyl)propanamide: 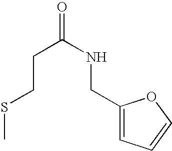
Compound 3: N-(4-methoxybenzyl)-3-(methylsulfanyl)propanamide: 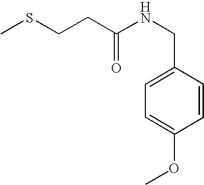
Compound 4: N-hexyl-3-(methylsulfanyl)propanamide: 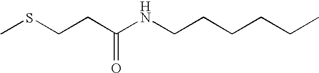
Compound 5: 3-(methylsulfanyl)-N-[2-(trifluoromethyl)phenyl]propanamide: 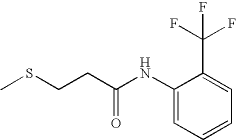
Compound 6: N-benzyl-3-(methylsulfanyl)propanamide: 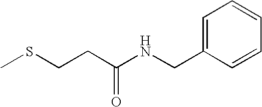
Compound 7: 3-(methylsulfanyl)-N-(1-phenylethyl)propanamide: 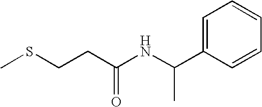
Compound 8: N-cyclobutyl-3-(methylsulfanyl)propanamide:

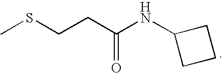

25. The topically applicable composition as defined by claim 23, formulated as a hair cream or hair lotion, a shampoo, a hair conditioner, a hair mascara or an eyelash mascara.

26. The topically applicable composition as defined by claim 23, further comprising at least one ingredient selected from the group consisting of solvents, aqueous-phase or oily-phase thickeners or gelling agents, dyestuffs that are soluble in the medium of the composition, fillers, pigments, antioxidants, sequestrants, preservatives, fragrances, electrolytes, neutralizers, film-forming polymers, UV blockers, cosmetic or pharmaceutical active agents other than the compounds of formula (I), and mixtures thereof.

27. The topically applicable composition as defined by claim 23, further comprising at least one active agent selected from the group consisting of proteins, protein hydrolysates, amino acids, polyols, urea, allantoin, sugars and sugar derivatives, water-soluble vitamins, plant extracts, hydroxy acids, retinol, tocopherol, retinol or tocopherol derivatives, essential fatty acids, ceramides, essential oils, salicylic acid derivatives, for instance, 5-n-octanoylsalicylic acid, hydroxy acid esters and phospholipids, and mixtures thereof.

28. The topically applicable composition as defined by claim 23, further comprising at least one additional active agent that promotes the regrowth and/or limits the loss of keratin fibers.

29. The topically applicable composition as defined by claim 28, further comprising at least one additional active agent selected from the group consisting of aminexil, diethyl 2,4-pyridinedicarboxylate, 6-0-[(9Z,12Z)-octadeca-9,12-dienoyl]hexapyranose, lipoxygenase inhibitors, bradykinin inhibitors, prostaglandins and derivatives thereof, prostaglandin receptor agonists or antagonists, non-prostanoic prostaglandin analogs, vasodilators, anti-androgens, cyclosporins and analogs thereof, anti-microbial agents, anti-inflammatory agents, retinoids, benzalkonium chloride, benzethonium chloride, phenol, oestradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharidic or acylhexosaccharic acids, substituted arylethylenes, N-acylamino acids, flavonoids, ascomycin derivatives and analogs, histamine antagonists, saponins, proteoglycanase inhibitors, oestrogen agonists and antagonists, pseudoterines, cytokines and growth factor promoters, IL-1 or IL-6 inhibitors, IL-10 promoters, TNF inhibitors, vitamins, benzophenones, hydantoin, retinoic acid, anti-pruriginous agents, anti-parasitic agents, anti-fungal agents, calcium antagonists, hormones, triterpenes, anti-androgens, steroidal or non-steroidal 5-α-reductase inhibitors, potassium channel agonists and FP receptor agonists, and mixtures thereof.

30. A 3-sulfanylpropanamide compound of one of the formulae (III), (IV), (V), (VII) and (VIII) or a salt or solvate thereof: 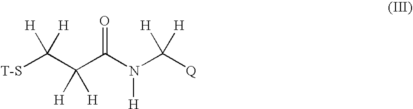
wherein Q is:
a) a phenyl radical substituted with at least one radical Z; or
b) an aromatic heterocycle selected from the group consisting of pyrimidine, quinoline, isoquinoline, pyrrole, furan or thiophene, optionally substituted with at least one radical Z,
wherein Z is:
fluorine,
a group CF₃, CN, OR, SR, NRR′, SO₂NRR′, NRSO₂R′, SO₂R, SiRR′R″,
a group of formula (I1): 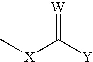
wherein:
W=O, NH, S,
X=O, NH, C₁-C₆ N-alkyl,
Y=C₁-C₄ alkyl, C₁-C₆ alkoxy, NH₂, C₁-C₆ NH-alkyl, C₁-C₆ N-dialkyl,
a C₁-C₁₀ alkyl radical optionally substituted with at least one substituent A₄;
R, R′ and R″, which may be identical or different, are each:
1) a hydrogen atom,
2) a saturated C₁-C₆ alkyl radical optionally substituted with at least one substituent A₄, or
3) an unsaturated hydrocarbon-based ring C₅ optionally substituted with at least one substituent A₄,
4) a heterocycle Hy₂ comprising at least one hetero atom selected from among N, S and O or a combination thereof and optionally being substituted with at least one substituent A₄,
wherein A₄ is:
1) a fluorine atom,
2) a group CF₃, CN, OR, SR, NRR′
3) a C₁-C₂₀ alkyl group, or
4) a ring C⁶ optionally fused to another ring C⁷, such rings optionally containing at least one hetero atom to form a heterocycle Hy₂,
wherein T is:
a) a saturated alkyl radical selected from among methyl, ethyl, n-butyl, -isobutyl and tert-butyl; or
b) a saturated C₄-C₆ hydrocarbon-based ring;

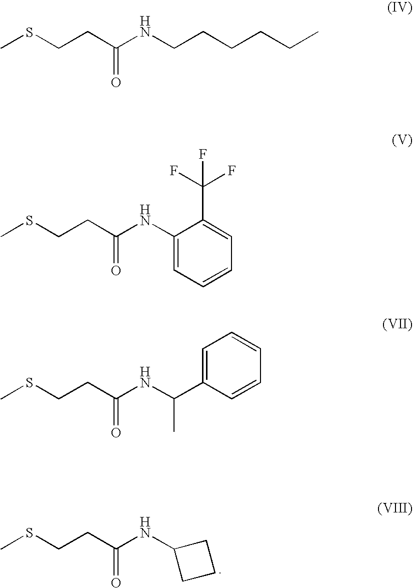

31. A 3-sulfanylpropanamide compound as defined by claim 30, selected from the group consisting of:
Compound 1: N-[(5-methyl-2-furyl)methyl]-3(methylsulfanyl)propanamide: 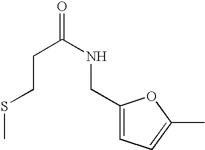
Compound 2: N-(2-furylmethyl)-3-(methylsulfanyl)propanamide: 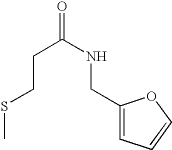
Compound 3: N-(4-methoxybenzyl)-3-(methylsulfanyl)propanamide: 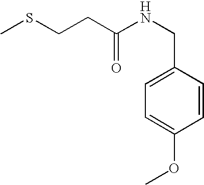
Compound 4: N-hexyl-3-(methylsulfanyl)propanamide: 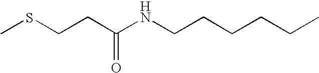
Compound 5: 3-(methylsulfanyl)-N-[2-(trifluoromethyl)phenyl]propanamide: 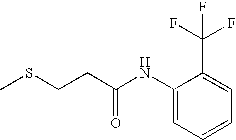
Compound 6: N-benzyl-3-(methylsulfanyl)propanamide: 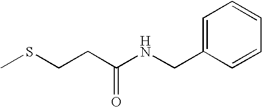
Compound 7: 3-(methylsulfanyl)-N-(1-phenylethyl)propanamide: 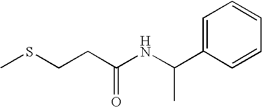
Compound 8: N-cyclobutyl-3- (methylsulfanyl)propanamide: [chemical structure]
